(19) United States
(12) Patent Application Publication
(10) Pub. No.: US 2022/0251177 A1
(43) Pub. Date: Aug. 11, 2022

(54) METHODS AND COMPOSITIONS RELATING TO ANTI-NUCLEOLIN RECOMBINANT IMMUNOAGENTS

(71) Applicants: OHIO STATE INNOVATION FOUNDATION, Columbus, OH (US); CEINGE-Biotecnologie Avanzate s.c.a. r.l., Naples (IT)

(72) Inventors: Dario PALMIERI, Columbus, OH (US); Claudia DE LORENZO, Napoli (IT); Carlo CROCE, Columbus, OH (US)

(21) Appl. No.: 17/483,101

(22) Filed: Sep. 23, 2021

Related U.S. Application Data
(63) Continuation of application No. 15/741,729, filed on Jan. 3, 2018, now abandoned.
(60) Provisional application No. 62/190,855, filed on Jul. 10, 2015.

(51) Int. Cl. C07K 16/18; G01N 33/574; G01N 33/50; A61K 39/395; A61K 45/06; C12N 15/113; A61K 47/68; A61P 35/00; A61K 49/00

(52) U.S. Cl. CPC C07K 16/18; A61K 2039/505; G01N 33/50; A61K 39/395; G01N 33/574; A61K 45/06; C12N 15/113; A61K 47/6843; A61K 47/6807; A61P 35/00; A61K 49/0004; G01N 2800/52; C07K 2317/92; C07K 2317/77; C07K 2317/73; C07K 2317/76; G01N 33/57496

(57) ABSTRACT

Disclosed herein are methods and compositions related to single chain antibody fragments which specifically bind nucleolin (NCL). Also disclosed are treating and diagnosing diseases using single chain antibody fragments that bind nucleolin.

### CROSS-REFERENCE TO RELATED APPLICATIONS

This application claims benefit of U.S. Provisional Application No. 62/190,855, filed Jul. 10, 2015, which is hereby incorporated herein by reference in its entirety.

### BACKGROUND

Nucleolin (NCL) is one of the most abundant non-ribosomal proteins in the nucleolus (Bugler et al. FEBS 128(2-3):475-480), first identified in ribosomal RNA processing (Warner, Current opinion in cell biology 2(3):521-527). Further studies have demonstrated that NCL is a multifunctional nucleocytoplasmic protein, involved in ribosomal assembly, chromatin decondensation, transcription, nucleo-cytoplasmic import/export and chromatin remodeling (Borer et al Cell 56(3):379-390; Mongelard et al. Trends in cell biology 17(2):80-86). NCL is frequently up-regulated in cancer and in cancer-associated endothelial cells compared to normal tissues (Srivastava et al. FASEB journal: 13(14):1911-1922; Ridley L, et al. Neuro-oncology 10(5):675-689), where it is also present on the cell surface (Hovanessian A G, et al. (2000) Experimental cell research 261(2):312-328, Christian S, et al. (2003) JCB 163(4):871-878). Altered NCL expression and localization results in oncogenic effects such as stabilization of AKT, Bcl-2, Bcl-XL, and IL-2 mRNAs (Otake Y, et al. (2007) Blood 109(7):3069-3075; Chen C Y, et al. (2000) Genes & development 14(10):1236-1248, Abdeimohsen K, et al, (2011) NAR 39(19):8513-8530). Moreover, surface-NCL acts as a receptor for several oncogenic ligands (Reyes-Reyes E M & Akiyama S K (2008) Experimental cell research 314(11-12):2212-2223; Tate A, et al. (2006) BMC cancer 6:197; Wise J F, et al. (2013) Blood 121(23):4729-4739; Abdelmohsen K & Gorospe M (2012) RNA Biology 9(6):799-808) and viruses (Tayyari F, et al. (2011) Nat. Med. 17(9):1132-1135).

NCL has a critical pro-tumorigenic function regulating the biogenesis of selected microRNAs (miRNAs), a class of non-coding single stranded RNAs 19-22-nt in length (Bartel D P (2004) Cell 116(2):281-297) which regulate gene expression at the post-transcriptional level by targeting mRNAs in a sequence specific manner (Pillai et al. Trends in cell biology 17(3):118-126). In fact, NCL enhances the maturation of specific miRNAs (including miR-21, miR-221 and miR-222) causally involved in cancer pathogenesis and resistance to several anti-neoplastic treatments (Pichiorri F, et al. (2013) The Journal of Experimental Medicine 210(5):951-968; Rao X, et al. (2011) Oncogene 30(9):1082-1097; Pogribny I P, et al, (2010) Int'l Jnl Cancer 127(8):1785-1794; Anastasov N, et al. (2012) Radiation Oncology 7:206; Mei M, et al. (2010) Technology in Cancer Research & Treatment 9(1):77-86). These findings demonstrated that NCL modulates the biogenesis of these miRNAs at the post-transcriptional level, enhancing their maturation from pri- to pre-miRNAs, identifying a novel NCL-dependent oncogenic mechanism (Pichiorri F, et al. (2013) The Journal of Experimental Medicine 210(5):951-968).

Because of its oncogenic role and specific expression on cancer cells surface, NCL represents an attractive target for anti-neoplastic therapies (Bates et al. (2009) Experimental and Molecular Pathology 86(3):151-164). Several groups have attempted to develop molecules such as aptamers (AS1411) or peptides (HB-19, V3 loop-mimicking pseudopeptide, N6L and F3) (Bates et al. (2009) Experimental and Molecular Pathology 86(3):151-164; Koutsioumpa M & Papadiinitriou E (2013); Destouches D, et al. (2008) PloS one 3(6):e2518; El Khoury D, et al. (2010) BMC cancer 10:325; Krust et al. (2011) BMC cancer 11:333) to bind and to inhibit NCL in cancer cells. These compounds have also been suggested as potential carriers for the targeted delivery into cancer cells of several anti-neoplastic agents (Bates et al. (2009) Experimental and Molecular Pathology 86(3):151-164). Although promising, aptamers and peptides targeting NCL suffer from intrinsic limitations, such as extremely short half-life, undesired immunostimulatory actions and still unknown toxicological effects (Abdelmohsen et al. RNA biology 9(6):799-808). What is needed in the art is a fully human anti-NCL immune-based agent displaying anti-neoplastic activity against solid tumors, both in vitro and in vivo.

### SUMMARY

Disclosed herein is an antibody fragment which specifically binds nucleolin (NCL). The fragment can be a single chain Fragment variable fragment (scFv), for example. The antibody fragment can specifically bind to the RNA binding domain (RBD) of nucleolin.

Also disclosed is a method for in vivo treatment of a mammal having NCL-expressing cancer comprising a step of administering to the mammal a therapeutically effective amount of a composition comprising a scFv that specifically binds NCL.

Further disclosed is a method for in vitro immunodetection of nucleolin-expressing cancer cells comprising a step of contacting the cancer cells with a composition comprising a scFv that specifically binds nucleolin.

Disclosed is a method of in vivo immunodetection of NCL-expressing cancer cells in a mammal comprising a step of administering to the mammal a diagnostically effective amount of a composition comprising a scFv that specifically binds nucleolin.

Also disclosed is a method of in vivo treatment of cancer comprising the steps of: (a) intravenously administering a radionuclide-labeled antibody fragment, wherein said antibody fragment binds nucleolin; (b) thereafter detecting tumor cells using a radionuclide activity probe; and (c) thereafter removing the detected tumor cells by surgical excision.

Disclosed herein is a kit comprising the antibody fragment that specifically binds nucleolin.

Disclosed herein is a method of making an antibody fragment, comprising: (a) culturing art isolated bacterial cell, wherein said cell is capable of producing a scFv specific for NCL, under conditions such that said antibody fragment is expressed; and (b) recovering said antibody fragment from the cell.

Disclosed is a method of treating cancer comprising administering to a subject in need thereof a composition comprising a scFv that specifically binds nucleolin, wherein the effector moiety is a chemotherapeutic agent.

Disclosed is a method for prognosing recurrence of cancer in a subject previously treated for cancer, the method comprising: (a) isolating a biological sample comprising cells from a subject with a cancer; (b) contacting the biological sample with a compositions comprising an antibody fragment that binds nucleolin under conditions sufficient for the composition to bind to an epitope present on a tumor and/or a cancer cell, if present, in the biological sample; and (c) identifying in the biological sample one or more cells that bind to the composition comprising an antibody that specifically binds NCL, whereby recurrence of a cancer is prognosed in the subject.

Also disclosed are methods for diagnosing cancer in vivo, for example. This can be done through the use of a scFv labeled with fluorescent or radioactive compounds and isotopes. Also disclosed are scFvs with chemical alterations, including PEGylation or discrete PEGylation of the scFv.

### DESCRIPTION OF DRAWINGS

FIG. 1 shows selection and purification of human anti-NCL scFvs by phage display. (A) Binding of selected phage clones or soluble scFvs to NCL was assessed by ELISA using NCL-coated plates incubated with the indicated clones. Clone 4LB5 used for further experiments is indicated (*). The assay was performed three times in triplicate using different preparation of phages and scFvs, and mean±SD is reported. (B) Selected Clone named 4LB5 was subcloned in PET22b E. coli expression vector and transformed in BL21-DE3 bacterial cells. (C) IPTG-induced scFv was extracted from inclusion bodies, than refolded using urea gradient, and finally purified using Ni-NTA columns, M=Molecular Standard; 1-6=Different elutions of the scFv; FT=Flow Through.

FIG. 2 shows anti-NCL scFv 4LB5 specifically binding to NCL in vitro and on cancer cell surface. (A) 4LB5 affinity for recombinant NCL was assessed by ELISA using different amounts of scFv. Apparent K_{d} is also indicated. Curve equation and R² are also reported. (B) ELISA assay performed on MDA-MB-231 using different amounts of 4LB5. Curve equation and R² are also reported. (C) ELISA assay performed using different amounts of 4LB5 on MDA-MB-231 cells following control (siCTRL) or anti-NCL (siNCL) siRNA transfection, *p<0.05; **p<0.01. (D) ELISA assay on MCF-10a (surface-NCL negative) and MDA-MB-231 (surface-NCL positive) breast cancer cells. **p<0.01. All the experiments are representative of three independent experiments performed in triplicate. Mean+/−SD is reported.

FIG. 3 shows kinetic evaluation of 4LB5 binding to recombinant NCL and 4LB5 specific binding to NCL. (A) 4LB5 affinity for recombinant NCL-RBD was assessed by Surface Plasmon Resonance using increasing concentrations (1-50 nM) of scFv. Resulting K_{d} is also indicated. (B) The detection limit of ELISA assay using 4LB5 (FIG. 2) was assessed using different amounts of scFv and indicated numbers of MDA-MB-231 cells. Data (normalized for background levels) are representative of two independent experiments performed in triplicate, ±SD. **′p<0.01, compared to the corresponding negative control. (C) Specific binding of 4LB5 to NCL assessed by Western Blot on MDA-MB-231 cells transfected with control (siCTRL) or anti-NCL (siNCL) siRNA. 4LB5 was used as a primary antibody and HRP-conjugated anti-His6 as secondary antibody. Bands indicated by the asterisk are due to the secondary anti-His6 antibody used for the detection of the NCL-bound scFv. Tubulin was used as a loading control.

FIG. 4 shows 4LB5 binds NCL on the surface of different cancer cell lines. Indicated cell lines (A, MCF-10a Normal-Like Breast; B, MDA-MB-436 Basal B TNBC; C, BT-549 Basal B TNBC; D, Huh7 HCC; E, MDA-MB-231 Basal B TNBC; F, T47D Luminal Breast Cancer; G, PLC-PRF, HCC) were stained or not for 1 hour with 2 μg/ml Cy5.5-Labeled 4LB5 and analyzed by flow cytometry. Mean Fluorescent Index (MFI) is also reported in parenthesis. Data are representative of three independent experiments performed in duplicate.

FIG. 5 shows heterogeneous levels of surface NCL on the cancer cell lines used in the study. Indicated cell lines were stained using a commercially available anti-NCL antibody and analyzed by flow cytometry using the FlowJo software. The relative abundance of different subpopulations, expressing different levels of surface NCL, is reported.

FIG. 6 shows 4LB5 is internalization by target cells. MDA-MB-231 cells were incubated for 6 hours at 37° C. (A) or 4° C. (B) with Cy5-labeled 4LB5. Cells were then harvested and analyzed using a FlowSight instrument (AMNIS) to acquire Bright Field (Ch01), Cy5 (Ch11), and merged images. At least 10,000 cells were acquired for each experimental point. (C-D) Internalization analysis was performed using the FlowSight Internalization wizard and quantification of cells internalizing 4LB5 in the two conditions is reported.

FIG. 7 shows anti-NCL 4LB5 scFv inhibition of microRNA biogenesis. (A) HeLa cells, trartsfected as indicated, were incubated for 24 h with or without 120 nM 4LB5. The interaction between myc-tagged NCL and FLAG-tagged DGCR8 was assessed by co-immunoprecipitation followed by SDS-PAGE and western blot. Control IgG was used as negative control to evaluate the specificity of the anti-Myc antibody for the immuno-precipitation. (B) REMSA performed by incubating recombinant NCL and biotin-labeled miR-21 in the presence of increasing amounts (80-650 nM) of 4LB5. Control IgG was used as negative control (CTRL). (C-D) NCL-dependent microRNA levels were analyzed by Real-Time PCR 72 h following 15 nM 4LB5 treatment. Both mature (C) and primary (D) microRNA levels were analyzed. Data are the average of three independent experiments performed in triplicate. *p<0.05; **p<0.01.

FIG. 8 shows 4LB5 affecting cancer cell proliferation and survival. (A) Basal B Triple Negative Breast Cancer (TNBC) cells MDA-MB-231 were treated with increasing amounts of 4LB5. Viable cells were counted using trypan blue staining at different time points (Light blue squares, 24 hours; red triangles 48 hours; green circles, 72 hours). All the experiments are representative of four independent experiments performed in triplicate. Mean+/−SD is reported. *p<0.05; **p<0.01; ***p<0.001 (B) Growth curves performed on MDA-MB-231 cells left untreated or treated with 30 nM or 120 nM 4LB5. Total cells were counted at the indicated time points. All the experiments are representative of three independent experiments performed in triplicate. Mean+/−SD is reported. *p<0.05; **p<0.01; ***p<0.001. (C) Representative images of the cells shown in (A), treated for 72 hours as indicated. Bars indicate 100 um. (D) Colony assay on MDA-MB-231 cells treated with or without 30 nM 4LB5, and stained after 10 days with crystal violet. All the experiments are representative of three independent experiments performed in triplicate.

FIG. 9 shows 4LB5 affecting cancer cell survival, Indicated (A, T47D; B, BT-549; C, MDA-MB-436; D, PLC-PRF; E, Huh7; F, MCF-10a) were treated with increasing amount of 4LB5. Viable cells were counted using trypan blue staining at different time points (24, 48 and 72 hours). All the experiments are representative of three independent experiments performed in quadruplicate. Mean±SD is reported. ***p<0.001.

FIG. 10 shows 4LB5 cytotoxic effect dependent on surface-NCL expression and is prevented by overexpression of specific microRNAs. (A) MDA-MB-231 cells were transfected with control (siC) or anti-NCL (siNCL) siRNAs for 24 hours, and then untreated or treated with 30 nM 4LB5 for 48 hours. Total cells were counted. Data are representative of three independent experiments performed in quadruplicate. Mean±SD is reported. **p<0.01. (B) MDA-MB-231 cells were transfected with scramble RNA or indicated mature microRNAs for 24 hours, and treated or not with 50 nM 4LB5 for 48 hours. Total cells were counted. Data are representative of two independent experiments performed in quadruplicate. Mean+/−SE is reported. *p<0.05.

FIG. 11 shows 4LB5 inhibition of cancer cell migration. Indicated cell lines were treated or left untreated for 24 h with 150 nM 4LB5, then counted and 5×10⁴ viable cells were plated in the presence or in the absence of the scFv in transwell chambers for additional 24 h. Following migration, cells were stained with crystal violet and acquired using a phase-contrast microscope. Data are representative of two independent experiments performed in triplicate.

FIG. 12 shows 4LB5-induced apoptosis. (A-B) Cell cycle analysis of MDA-MB-231 (A) and PLC-PRF (B) cells by propidium iodide staining, treated or not (NT, not treated) with 240 nM 4LB5 for 48 h and 72 h. Red peaks indicate cells in G1 and G2 phase of the cell cycle. Stripes indicate cells in S phase. Blu peaks indicate sub-G1 apoptotic cells. Data are representative of three independent experiments (C-D) Western Blot analysis of total lysates from cells treated as in (A-B) to evaluate inactive-PARP cleavage and AKT levels. GAPDH was used as loading control. (E) Caspase 3/7 activation assay performed on MDA-MB-231 and PLC-PRF cells 24 h following the treatment with 30 nM 4LB5 or control vector (NT). **, p<0.01. Data are representative of three independent experiments performed in triplicate.

FIG. 13 shows 4LB5-induced apoptosis. (A-B) Cell cycle analysis of MDA-MB-436 (A) and T47D (B) cells by propidium iodide staining, treated or not (NT, not treated) with 240 nM 4LB5 for 48 h and 72 h. Red peaks indicate cells in G1 and G2 phase of the cell cycle. Stripes indicate cells in S phase. Blue peaks indicate sub-G1 apoptotic cells. (C-D) Western Blot analysis of total lysates from cells treated as in (A-B) to evaluate inactive-PARP cleavage and AKT levels. GAPDH was used as loading control.

FIG. 14 shows 4LB5 inhibition of breast cancer cell growth in vivo. (A-D) NOD-SCID (n=8) mice were injected with 2×10⁶ MDA-MB-231-Luc cells into the mammary fat pad. After 2 weeks mice were treated with control solution (n=4) (A and C) 2 mg/kg of 4LB5 (n=4) (B and D), twice a week. Mice were monitored by IVIS weekly. At 4 weeks from injection (2 weeks of treatment) mice were euthanized. Tumors were excised and measured. Bars indicate 1 cm. (E) Average volume for the tumors reported in A-D (L×W×H) is reported. *, p<0.05. (F) Representative images of H&E and Ki67 staining of tumors shown in (C-D) 20× magnification is reported. Bars indicate 50 μm. See also FIG. 15, where a different batch of 4LB5 was used in a separate experimental setting.

FIG. 15 shows 4LB5 inhibition of breast cancer cell growth in vivo. NOD-SCID (n=10) mice were injected with 2×10⁶ MDA-MB-231-Luc cells into the mammary fat pad. After 2 weeks, mice were treated with control solution (n=5) or 2 mg/kg of 4LB5 (n=5), twice a week. Mice were monitored by IVIS weekly. At 4 weeks from injection (2 weeks of treatment) mice were euthanized. Tumors were excised and measured. Distribution of the tumor volumes (L×W×H) is reported. *, p<0.05. See also FIG. 14, where a different batch of 4LB5 was used in a separate experimental setting.

FIG. 16 shows 4LB5 inhibition of breast cancer cell growth in vivo (2). (A-B) NOD-SCID mice (n=10) were injected with 2×10⁶ MDA-MB-231-Luc cells into the mammary fat pad. After 2 days, mice were treated with 2 mg/kg (n=5) of 4LB5 or with control solution (n=5), twice a week. At 4 weeks of treatment, mice were euthanized. Tumors were excised and measured. (C-D) Average tumor volume (L×W×H) (C) and weight (D) of tumors in (A-B) is reported. **, p<0.01. (E) Following euthanization, body weight was measured to evaluate potential toxic effects of the treatment.

FIG. 17A-C shows 4LB5 specifically binds NCL on the surface of melanoma cells in vitro. FIG. 17A shows SKMEL147 (human melanoma cells) or NL145 (mouse melanoma cells) analyzed by cell surface ELISA using increasing amounts of 4LB5. *, p<0.05 compared to the negative control (0 nM) stained cells. FIGS. 17B-C show binding of 4LB5 to surface NCL was assessed by cell surface ELISA (FIG. 17B) following control siRNA (siC) or anti-NCL siRNA (siNCL) transfection (assessed by Western Blot, shown in FIG. 17C. *, p<0.05 compared to the control siRNA transfected cells. Binding was assessed using two different concentrations of 4LB5 (10-100 nM).

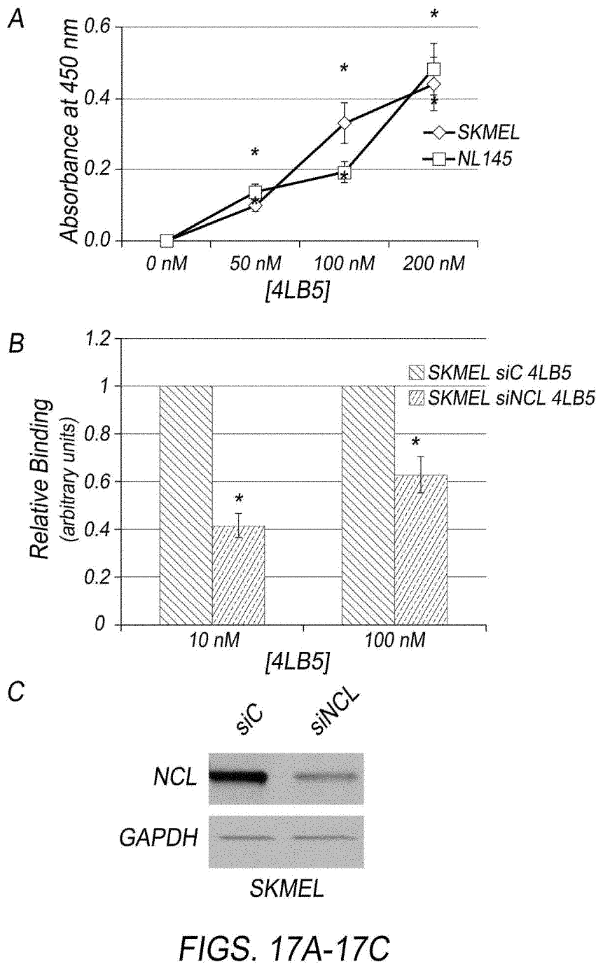

FIG. 18A-C shows 4LB5 specifically inhibits melanoma cell proliferation in vitro. FIG. 18A shows SKMEL147 (human melanoma cells) or NL145 (mouse melanoma cells) were seeded in 6-well plates (100 cells/well.) and were left untreated (NT) or treated with increasing amounts (10, 50 and 100 nM) of 4LB5. Resulting colonies were stained 7 days following the treatment using crystal violet and counted. *, p<0.05; **, p<0.01 compared to the non treated control. FIGS. 18B-C show KMEL147 (B) or NL-145 (C) cells were plated in 96-well plates, left untreated (NT) or treated using the indicated amounts of 4LB5. Proliferation was assessed by alamar blue assay at 48 h following the treatment. *, p<0.05; **, p<0.01 compared to the non treated controls. Data were normalized for the non treated controls.

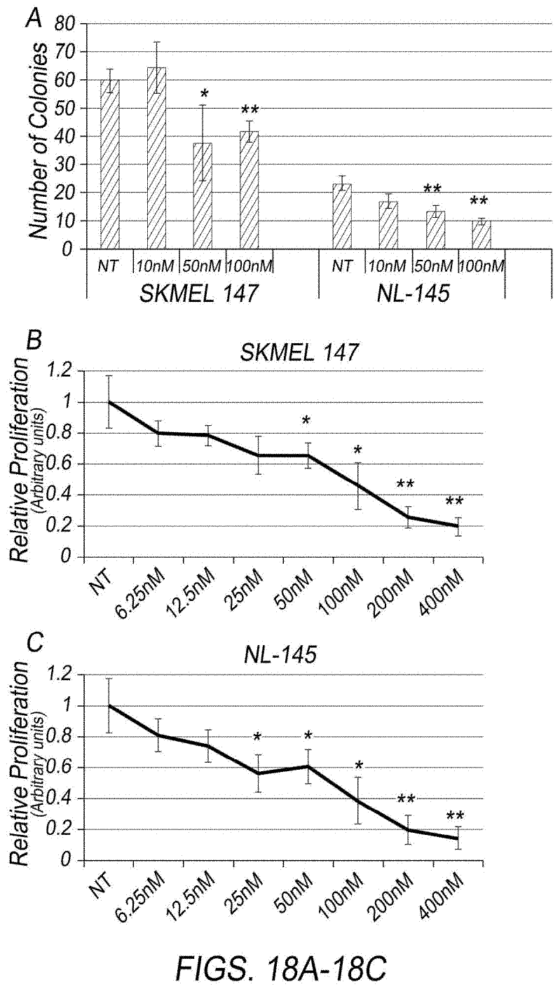

FIG. 19 shows 4LB5 inhibits UV-induced squamous cell carcinomas in a Skh-1 hairless model. Mice (n=4) were daily exposed to UV-light for 15 weeks to induce squamous cell tumors of the skin. Mice were then left untreated (n=2, left and middle-left) or i.p. injected with 2 mg/kg of 4LB5 (n=2, middle-right and right), twice per week for 10 more weeks (total 25 weeks). Tumor number, burden and total volume is reported for each mouse at the end of the treatment protocol.

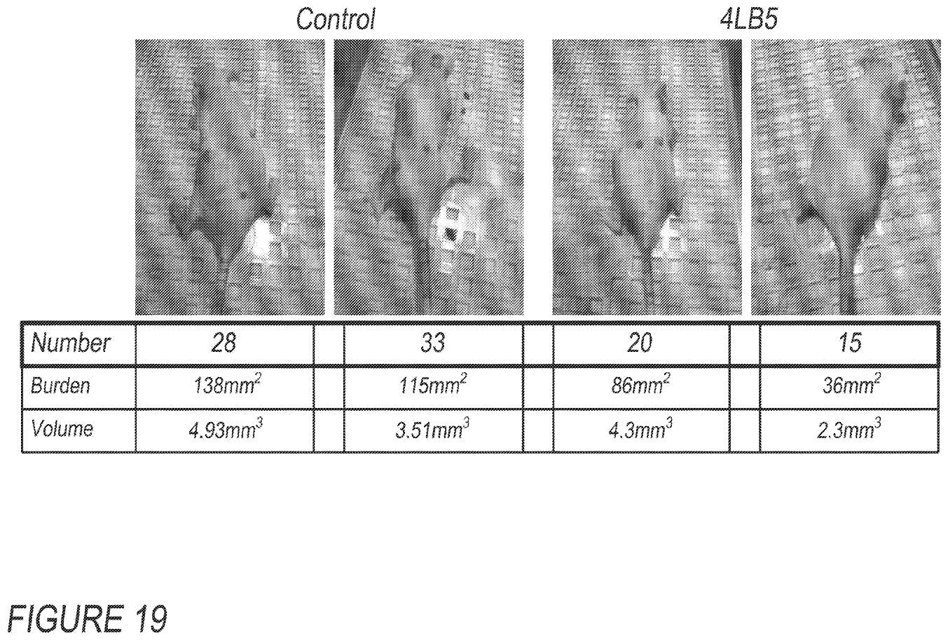

FIG. 20A-C shows 4LB5 radio-labeling. FIG. 20A shows SDS-PAGE following mock-labeling of 4LB5 using iodogen tube to show the integrity of 4LB5 following the treatment (no degradation observed, BSA was used as internal quantification control). Mock-labeled 4LB5 is indicated as 4Lb5\hat{ }. FIG. 20B shows Cell ELISA using 2 different cell lines probed with 4LB5 or 4LB5\hat{ }, displaying only a mild reduction in binding ability of 4LB5\hat{ } compared to 4LB5 following mock radiolabeling. FIG. 20C shows a Western blot of 4LB5\hat{ } before (pre load) or after its purification using an exclusion chromatography system.

FIG. 21 shows 4LB5 binds to the surface of lung cancer cells (H1299) in a NCL-dependent manner. Cell Surface ELISA shows an increase binding of 4LB5 with increasing concentration. It also shows decreased binding when transfected with siNCL compared to siCTRL-A.

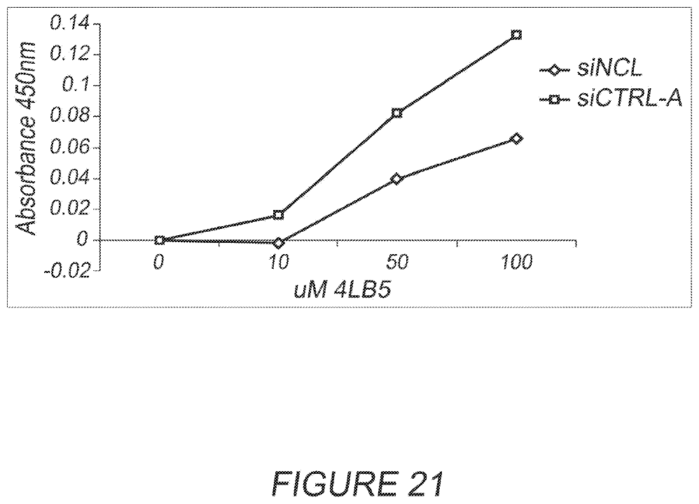

FIG. 22 shows cytotoxicity: 4LB5 inhibits lung cancer cell viability and proliferation. Cell survival curves: Cells were treated with concentrations of 4LB5 ranging from 0.5-512 nM, Calculation of IC50 values were performed using Graphpad Prism 6.

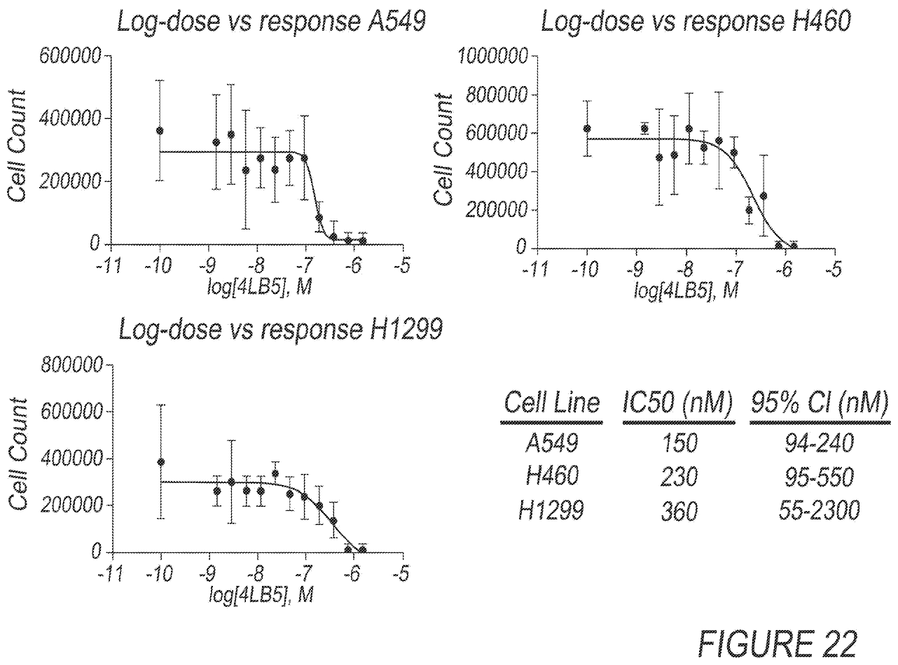

FIG. 23 shows that 4LB5 decreases NCL-dependent microRNA processing in various microRNAs.

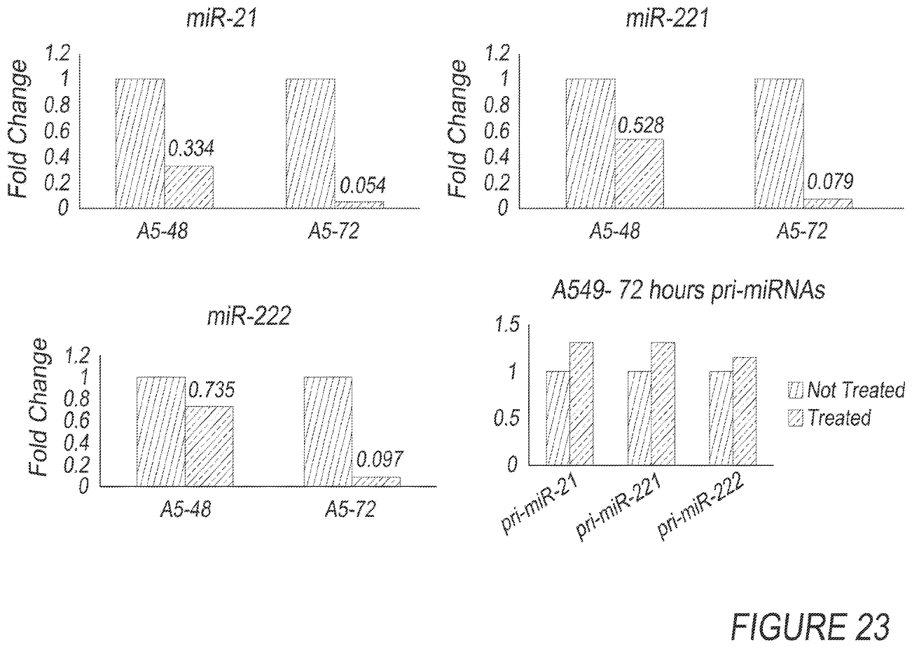

FIG. 24 shows a schematic of siRNA/miRNA delivery using 4LB5.

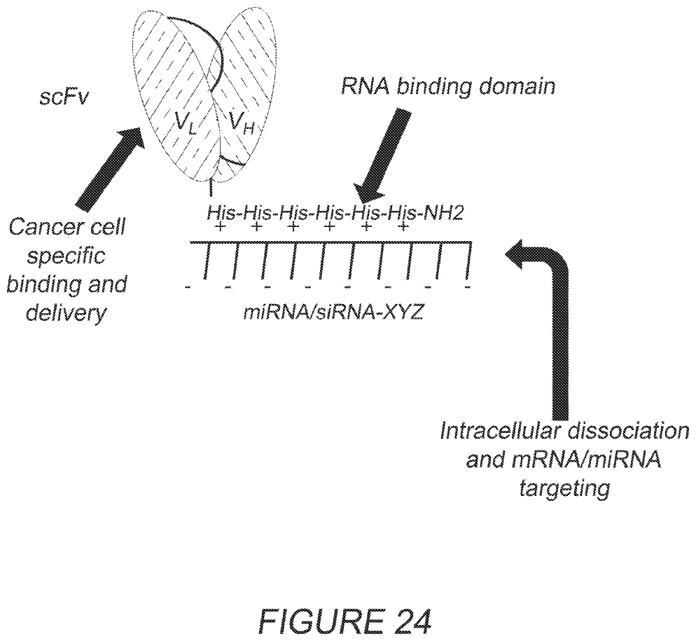

FIG. 25 shows 4LB5 miRNA conjugation using a REMSA assay. This assay was performed to demonstrate the effective binding ability of 4LB5 to microRNAs (in this case, miR-21). Biotinylated miR-21 was incubated with increasing amounts of 4LB5 and the complex was run on a non denaturing gel. The upper band corresponds to the microRNA/4LB5 complex (only observed in the presence of both, stronger and stronger with the increase of 4LB5 concentration, displaced by a molecular excess of non-biotinylated miR-21 or when an antibody against His-Tag is used).

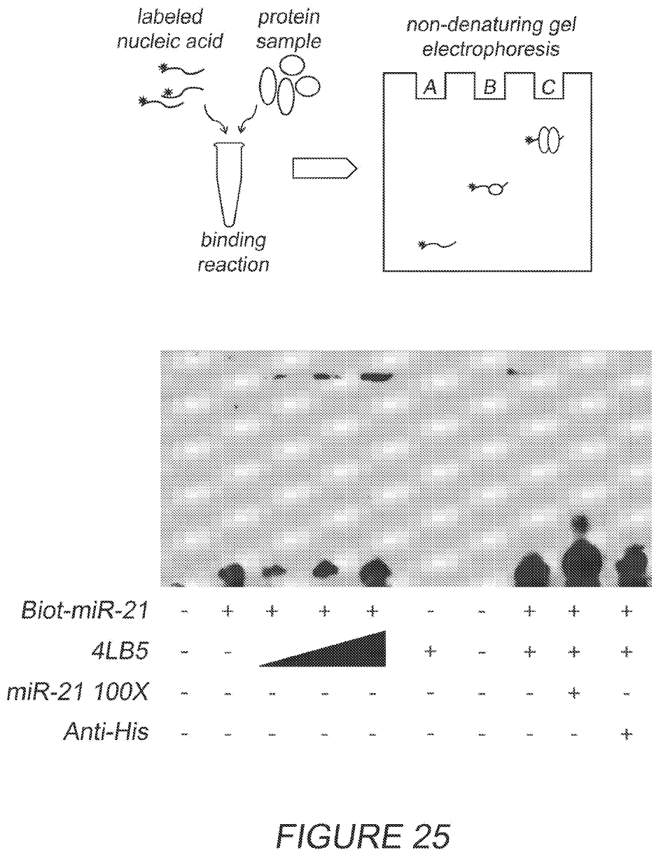

FIG. 26 shows miR-135b delivery. The conjugation between miR-135b and 4LB5 was performed for 15 minutes or O/N. The conjugate was used to treat breast cancer cells for 4 hours. Specific cell type used (as indicated) does not present miR-135b gene (removed by CRISPR/Cas9). RNA was extracted and Real Time PCR was performed. As controls, miR-135b alone (not conjugated) or 4LB5 conjugated with a scramble miRNA (at two different time points) were used. Equimolar amounts of 4LB5 and microRNAs were used for all conjugation experiment.

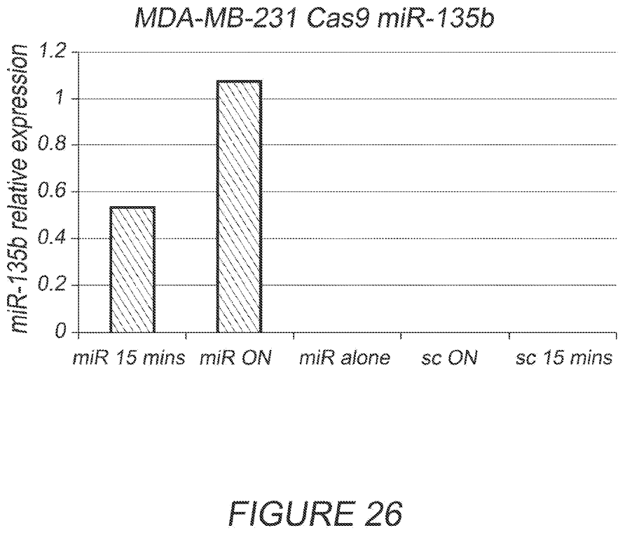

FIG. 27 shows non-human Ath-miR-159a miRNA delivery. Similarly to the experiment shown in FIG. 26, breast (MDA-MB-231) and lung (H1299) cells were treated with 4LB5 conjugated to a non-human microRNA (Ath-miR-159a) to demonstrate the generic ability to bind RNA sequences. Cells were harvested and RNA was extracted at different time points, as indicated, and Ath-miR-159a expression was evaluated by Real Time PCR.

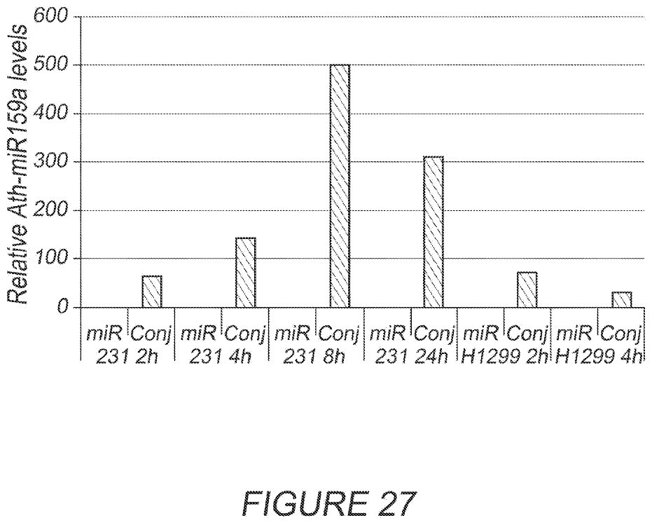

FIG. 28 shows in vivo miRNA delivery. Orthotopic mouse models of human breast cancer were i.p. treated with miR-16-1-conjugated 4LB5 (2 mg/kg) (n=1) or equivalent amount of miR-16-1 alone (n=1). At 24 h from the treatment, tumors, livers and kidneys were harvested. Total RNA was extracted from these organs and miR-16-1 expression was evaluated by Real Time PCR. Increase in the amount of miR-16-1 was observed in the tumor. Only a mild increase was observed in the liver. The increase in the kidney can due to the clearance of the conjugate (<< than microRNA alone). On the conjugate-treated tumor, an in situ hybridization was performed using a miR-16-1 specific or a scramble probe. Punctate staining when using miR-16-1 specific probe indicate the intracellular accumulation of miR-16-1.

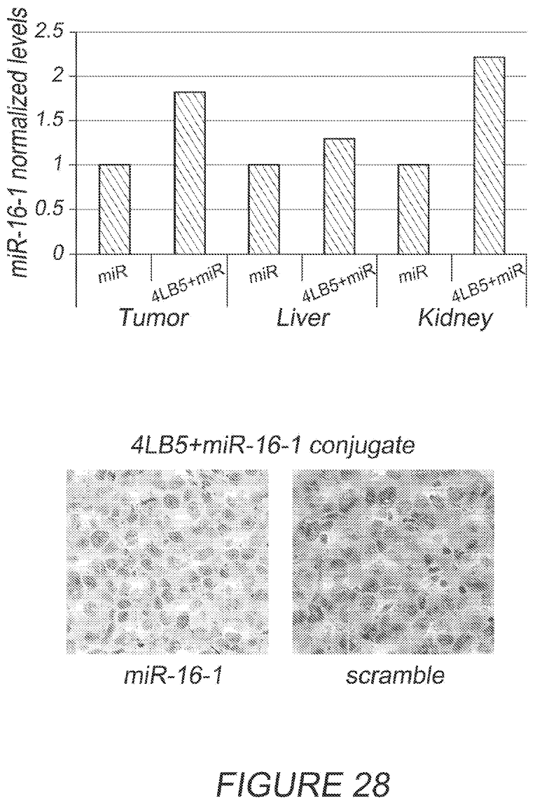

FIG. 29 shows evaluation of the up-regulation of PTEN expression and AKT phosphorylation following 4LB5 treatment by Western Blot, based on the widely described role of miR-221-PTEN-AKT pathway in the survival of different types of human tumors, including SC. Caspase 3/7 activation assay confirmed the 4LB5-dependent activation of apoptosis.

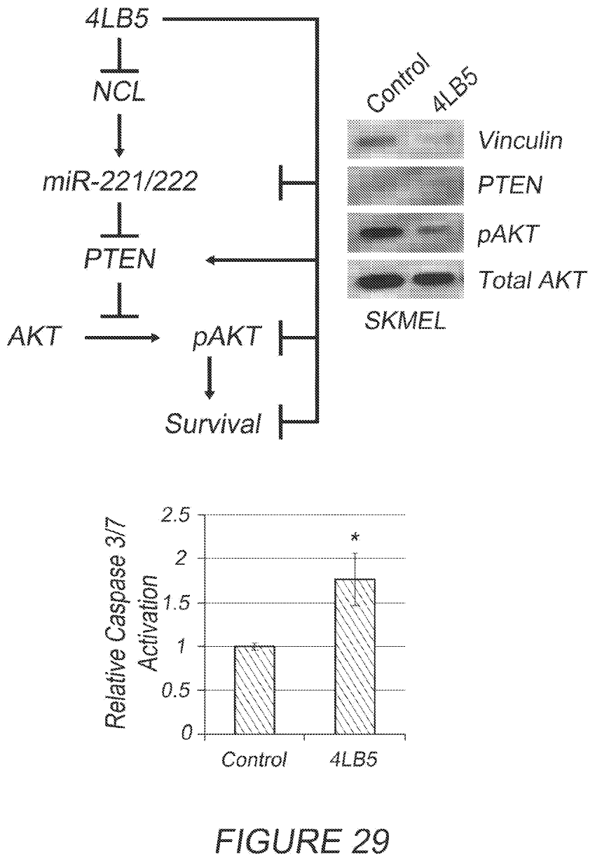

FIG. 30 shows NCL localization was verified by IHC on skin and tumors sections from control mice shown in FIG. 29, 4LB5 accumulation into scFv treated skin and tumors was also evaluated by IHC using anti-6His tag antibody.

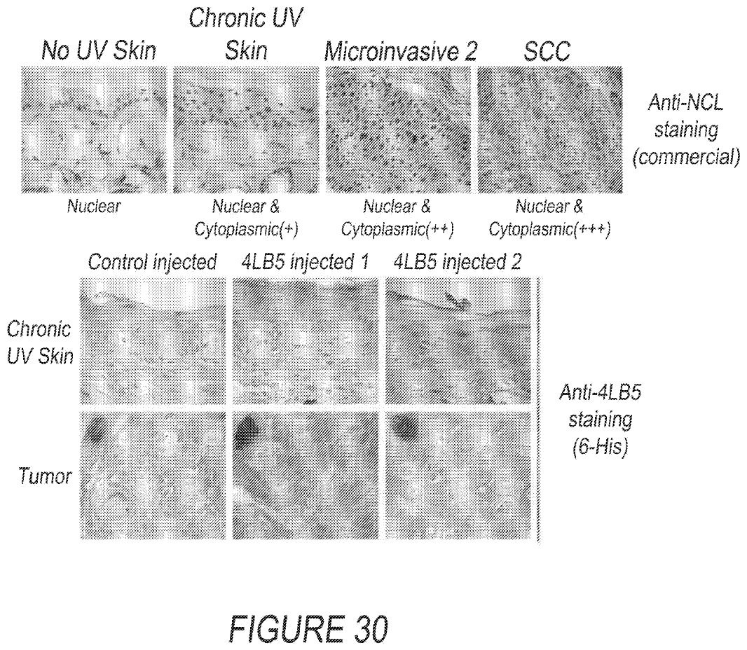

### DETAILED DESCRIPTION

The materials, compositions, and methods described herein can be understood more readily by reference to the following detailed descriptions of specific aspects of the disclosed subject matter and the Examples and Figure included herein.

Before the present materials, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

Definitions

Unless defined otherwise, all technical and scientific terms used herein have the same meaning as commonly understood by one of ordinary skill in the art. Methods and materials similar or equivalent to those described herein can be used in the practice or testing of the present disclosure. In this specification and in the claims that follow, reference will be made to a number of terms, which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “an antibody” includes mixtures of two or more such antibodies; reference to “the composition” includes mixtures of two or more such compositions, and the like.

“Optional” or “optionally” means that the subsequently described event or circumstance can or cannot occur, and that the description includes instances where the event or circumstance occurs and instances where it does not.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

As used herein, the term “and/or” when used in the context of a list of entities, refers to the entities being present singly or in combination. Thus, for example, the phrase “A, B, C, and/or D” includes A, B, C, and D individually, but also includes any and all combinations and sub combinations of A, B, C, and D.

With respect to the terms “comprising”, “consisting of”, and “consisting essentially of”, where one of these three terms is used herein, the presently disclosed and claimed subject matter can include the use of either of the other two terms. For example, in some embodiments, the presently disclosed subject matter relates to compositions comprising antibodies. It would be understood by one of ordinary skill in the art after review of the instant disclosure that the presently disclosed subject matter thus encompasses compositions that consist essentially of the scFv of the presently disclosed subject matter, as well as compositions that consist of the antibodies of the presently disclosed subject matter.

The term “subject” as used herein refers to a member of any invertebrate or vertebrate species. Accordingly, the term “subject” is intended to encompass in some embodiments any member of the Kingdom Animalia including, but not limited to the phylum Chordata (e.g., members of Classes Osteichythyes (bony fish), Amphibia (amphibians), Reptilia (reptiles), Ayes (birds), and Mammalia (mammals), and all Orders and Families encompassed therein. Specifically, the term “subject” can mean “human.” The term “subject” is used interchangeably with the term “patient.”

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

Similarly, all genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

The terms “cancer” and “tumor” are used interchangeably herein and can refer to both primary and metastasized solid tumors and carcinomas of any tissue in a subject, including but not limited to breast; colon; rectum; lung; oropharynx; hypopharynx; esophagus; stomach; pancreas; liver; gallbladder; bile ducts; small intestine; urinary tract including kidney, bladder, and urothelium; female genital tract including cervix, uterus, ovaries (e.g., choriocarcinoma and gestational trophoblastic disease); male genital tract including prostate, seminal vesicles, testes and germ cell tumors; endocrine glands including thyroid, adrenal, and pituitary; skin (e.g., hemangiornas and melanomas), bone or soft tissues; blood vessels (e.g., Kaposi's sarcoma); brain, nerves, eyes, and meninges (e.g., astrocytomas gliomas, glioblastomas, retinoblastomas, neuromas, neuroblastomas, Schwannomas and meningiomas). As used herein, the terms “cancer and “tumor” are also intended to refer to multicellular tumors as well as individual neoplastic or preneoplastic cells. In some embodiments, a cancer or a tumor comprises a cancer or tumor of an epithelial tissue such as, but not limited to a carcinoma.

As used herein in the context of molecules, the term “effector” refers to any molecule or combination of molecules whose activity it is desired to deliver/into and/or localize at a cell. Effectors include, but are not limited to labels, cytotoxins, enzymes, growth factors, transcription factors, drugs, etc.

As used herein in the context of cells of the immune system, the term “effector” refers to an immune system cell that can be induced to perform a specific function associated with an immune response to a stimulus. Exemplary effector cells include, but are not limited to natural killer (NK) cells and cytotoxic T cells (Tc cells).

As used herein, the term “expression vector” refers to a DNA sequence capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The construct comprising the nucleotide sequence of interest can be chimeric. The construct can also be one that is naturally occurring but has been obtained in a recombinant form useful for heterologous expression.

As used herein, the terms “operatively linked” and “operably linked” refer to transcriptional regulatory elements (such as, but not limited to promoter sequences, transcription terminator sequences, etc.) that are connected to a nucleotide sequence (for example, a coding sequence or open reading frame) in such a way that the transcription of the nucleotide sequence is controlled and regulated by that transcriptional regulatory element. Similarly, a nucleotide sequence is said to be under the “transcriptional control” of a promoter to which it is operably linked. Techniques for operatively linking a promoter region to a nucleotide sequence are known in the art.

As used herein, the term “prodrug” refers to an analog and/or a precursor of a drug (e.g., a cytotoxic agent) that substantially lacks the biological activity of the drug (e.g., a cytotoxic activity) until subjected to an activation step. Activation steps can include enzymatic cleavage, chemical activation steps such as exposure to a reductant, and/or physical activation steps such as photolysis. In some embodiments, activation occurs in vivo within the body of a subject.

Antibodies

As used herein, the terms “antibody” and “antibodies” refer to proteins comprising one or more polypeptides substantially encoded by immunoglobulin genes or fragments of immunoglobulin genes. Immunoglobulin genes typically include the kappa (κ), lambda (λ), alpha (a), gamma (γ), delta (δ), epsilon (ε), and mu (μ) constant region genes, as well as myriad immunoglobulin variable region genes. Light chains are classified as either κ or λ. In mammals, heavy chains are classified as γ, μ, α, δ, or ε, which in turn define the immunoglobulin classes, IgG, IgM, IgA, IgD, and IgE, respectively. Other species have other light and heavy chain genes (e.g., certain avians produced what is referred to as IgY, which is an immunoglobulin type that hens deposit in the yolks of their eggs), which are similarly encompassed by the presently disclosed subject matter. In some embodiments, the term “antibody” refers to an antibody that binds specifically to an epitope that is present on a tumor antigen.

A typical immunoglobulin (antibody) structural unit is known to comprise a tetramer. Each tetramer is composed of two identical pairs of polypeptide chains, each pair having one “light” chain (average molecular weight of about 25 kiloDalton (kDa)) and one “heavy” chain (average molecular weight of about 50-70 kDa). The two identical pairs of polypeptide chains are held together in dimeric form by disulfide bonds that are present within the heavy chain region. The N-terminus of each chain defines a variable region of about 100 to 1 10 or more amino acids primarily responsible for antigen recognition (sometimes referred to as the “paratope”). The terms variable light chain (VL) and variable heavy chain (VH) refer to these light and heavy chains, respectively.

Antibodies typically exist as intact immunoglobulins or as a number of well-characterized fragments that can be produced by digestion with various peptidases. For example, digestion of an antibody molecule with papain cleaves the antibody at a position N-terminal to the disulfide bonds. This produces three fragments: two identical “Fab” fragments, which have a light chain and the N-terminus of the heavy chain, and an “Fc” fragment that includes the C-terminus of the heavy chains held together by the disulfide bonds. Pepsin, on the other hand, digests an antibody C-terminal to the disulfide bond in the hinge region to produce a fragment known as the “F(ab)′2” fragment, which is a dimer of the Fab fragments joined by the disulfide bond. The F(ab)′2 fragment can be reduced under mild conditions to break the disulfide linkage in the hinge region, thereby converting the F(ab′)2 dimer into two “Fab” monomers. The Fab′ monomer is essentially an Fab fragment with part of the hinge region. With respect to these various fragments, Fab, F(ab′)2, and Fab′ fragments include at least one intact antigen binding domain (paratope), and thus are capable of binding to antigens.

Antibody fragments, as disclosed herein, can be also obtained using phage-display technology, which selects a molecule with immunological properties similar to the conventional antibodies, but not derived from real antibodies.

While various antibody fragments are defined in terms of the digestion of an intact antibody, one of skill will appreciate that these fragments (including, but not limited to Fab′ fragments) can be synthesized de novo either chemically or by utilizing recombinant DNA methodology. Thus, the term “antibody” as used herein also includes antibody fragments produced by the modification of whole antibodies and/or synthesized de novo using recombinant DNA methodologies. In some embodiments, the term “antibody” comprises a fragment that has at least one antigen binding domain (paratope). Antibody fragments can be also obtained using phage-display technology, which selects a molecule with immunological properties similar to the conventional antibodies, but not derived from real antibodies.

Antibodies can be polyclonal or monoclonal. As used herein, the term “polyclonal” refers to antibodies that are present together in a given collection of antibodies and that are derived from different antibody-producing cells (e.g., B cells). Exemplary polyclonal antibodies include, but are not limited to those antibodies that bind to a particular antigen and that are found in the blood of an animal after that animal has produced an immune response against the antigen. However, it is understood that a polyclonal preparation of antibodies can also be prepared artificially by mixing at least non-identical two antibodies. Thus, polyclonal antibodies typically include different antibodies that are directed against (i.e., bind to) the same and/or different epitopes (sometimes referred to as an “antigenic determinant” or just “determinant”) of any given antigen.

As used herein, the term “monoclonal” refers to a single antibody species and/or a substantially homogeneous population of a single antibody species. Stated another way, “monoclonal” refers to individual antibodies or populations of individual antibodies in which the antibodies are identical in specificity and affinity except for possible naturally occurring mutations that can be present in minor amounts. Typically, a monoclonal antibody (mAb or moAb) is generated by a single B cell or a progeny cell thereof (although the presently disclosed subject matter also encompasses “monoclonal” antibodies that are produced by molecular biological techniques as described herein). Monoclonal antibodies (mAbs or moAbs) are highly specific, typically being directed against a single antigenic site. Furthermore, in contrast to polyclonal antibody preparations, a given mAb is typically directed against a single epitope on the antigen.

In addition to their specificity, mAbs can be advantageous for some purposes in that they can be synthesized uncontaminated by other antibodies. The modifier “monoclonal” is not to be construed as requiring production of the antibody by any particular method, however. For example, in some embodiments, the mAbs of the presently disclosed subject matter are prepared using the hybridoma methodology first described by Kohler et al., 1975, and in some embodiments are made using recombinant DNA methods in prokaryotic or eukaryotic cells (see e.g., U.S. Pat. No. 4,816,567, the entire contents of which are incorporated herein by reference), mAbs can also be isolated from phage antibody libraries.

The antibodies, fragments, and derivatives of the presently disclosed subject matter can also include chimeric antibodies. As used herein in the context of antibodies, the term “chimeric”, and grammatical variants thereof, refers to antibody derivatives that have constant regions derived substantially or exclusively from antibody constant regions from one species and variable regions derived substantially or exclusively from the sequence of the variable region from another species.

The variable region allows an antibody to selectively recognize and specifically hind epitopes on antigens. That is the VL domain and VH domain, or subsets of the complementarity determining regions (CDRs) within these variable domains, of an antibody combine to form the variable region that defines a three dimensional antigen binding site. This quaternary antibody structure forms the antigen binding site present at the end of each arm of the antibody. More specifically, the antigen binding site is defined by three CDRs on each of the VH and VL chains. In some instances (e.g., certain immunoglobulin molecules derived from camelid species or engineered based on camelid immunoglobulins), a complete immunoglobulin molecule can consist of heavy chains only with no light chains.

In naturally occurring antibodies, there are six CDRs present in each antigen binding domain that are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops that connect, and in some cases form part of, the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable domain by one of ordinary skill in the art, since they have been precisely defined.

A particular kind of chimeric antibody is a “humanized” antibody, in which the antibodies are produced by substituting the CDRs of, for example, a mouse antibody, for the CDRs of a human antibody (see e.g., PCT International Patent Application Publication No. WO 1992/22653). Thus, in some embodiments, a humanized antibody has constant regions and variable regions other than the CDRs that are derived substantially or exclusively from the corresponding regions of a human antibody, and CDRs that are derived substantially or exclusively from a mammal other than a human.

The antibodies the presently disclosed subject matter can be single chain antibodies and single chain antibody fragments, such as single chain variable fragments. Single-chain antibody fragments contain amino acid sequences having at least one of the variable regions and/or CDRs of the whole antibodies described herein, but are lacking some or all of the constant domains of those antibodies. These constant domains are not necessary for antigen binding, but constitute a major portion of the structure of whole antibodies.

Single-chain antibody fragments can overcome some of the problems associated with the use of antibodies containing a part or all of a constant domain. For example, single-chain antibody fragments tend to be free of undesired interactions between biological molecules and the heavy-chain constant region, and/or other unwanted biological activities. Additionally, single-chain antibody fragments are considerably smaller than whole antibodies and can therefore be characterized by greater capillary permeability than whole antibodies, allowing single-chain antibody fragments to localize and bind to target antigen-binding sites more efficiently. Also, antibody fragments can be produced on a relatively large scale in prokaryotic cells, thus facilitating their production. Furthermore, the relatively small size of single-chain antibody fragments makes them less likely than whole antibodies to provoke an immune response in a recipient. The single-chain antibody fragments of the presently disclosed subject matter include, but are not limited to single chain fragment variable (scFv) antibodies and derivatives thereof such as, but not limited to tandem di-scFv, tandem tri-scFv, miniantibodies, and minibodies.

Fv fragments correspond to the variable fragments at the N-termini of immunoglobulin heavy and light chains. Fv fragments appear to have lower interaction energy of their two chains than Fab fragments. To stabilize the association of the VH and VL domains, they can be linked with peptides, disulfide bridges, and/or “knob in hole” mutations.

A “single-chain variable fragment” (scFv) is a fusion protein of the variable regions of the heavy (VH) and light chains (VL) of immunoglobulins, connected with a short linker peptide. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. scFv can be produced in bacterial cells such as E. coli or in eukaryotic cells.

Methods and Compositions

scFvs and Nucleic Acids Thereof

Nucleolin (NCL) is a nucleocytoplasmic protein involved in many biological processes, such as ribosomal assembly, rRNA processing, and mRNA stabilization. NCL also regulates the biogenesis of specific microRNAs (miRNAs) involved in tumor development and aggressiveness. Interestingly, NCL is expressed on the surface of actively proliferating cancer cells, but not on their normal counterparts. Therefore, NCL is an attractive target for anti-neoplastic treatments. Taking advantage of phage-display technology, a fully human single-chain Fragment variable (scFv) was engineered, referred to herein as 4LB5. This immunoagent binds NCL on the cell surface, it is translocated into the cytoplasm of target cells, and it abrogates the biogenesis of NCL-dependent miRNAs. Binding of 4LB5 to NCL on the cell surface of a variety of breast cancer and hepatocellular carcinoma cell lines, but not to normal-like MCF-10a breast cells, dramatically reduces cancer cell viability and proliferation. Finally, in orthotopic breast cancer mouse models, 4LB5 administration results in a significant reduction of the tumor volume without evident side effects.

Disclosed herein are scFvs, which specifically bind nucleolin (NCL). Even more specifically, they can bind the RNA binding domain (RBD) of NCL. These highly stable, high-affinity, bacterially-expressible scFvs are capable of specifically binding to RBD of NCL. For example, they can bind only to RBD, so that they are specific only for RBD and not for other domains of nucleolin. The antibodies disclosed herein can inhibit nucleolin, for example, by 5, 10, 15, 20, 25, 30, 35, 40, 45, 50, 55, 60, 65, 70, 75, 80, 85, 90, 95, or 100%. This inhibition of nucleolin can inhibit tumor growth. Growth inhibition can be indicated by reduced tumor volume or reduced occurrences of metastasis. Tumor growth can be determined, e.g., by examining the tumor volume via routine procedures (such as obtaining two-dimensional measurements with a dial caliper). Metastasis can be determined by inspecting for tumor cells in secondary sites or examining the metastatic potential of biopsied tumor cells in vitro using well-known techniques. Inhibiting nucleolin can also inhibit infection.

Examples of cancer cells that can be inhibited or killed by a human anti-nucleolin antibody include but are not limited to: Acute Lymphoblastic Leukemia; Myeloid Leukemia; Acute Myeloid Leukemia; Chronic Myeloid Leukemia; Adrenocortical Carcinoma Adrenocortical Carcinoma; AIDS-Related Cancers; AIDS-Related Lymphoma; Anal Cancer; Astrocytoma, Childhood Cerebellar; Astrocytoma, Childhood Cerebral; Basal Cell Carcinoma; Bile Duct Cancer, Extrahepatic; Bladder Cancer; Bladder Cancer; Bone Cancer, osteosarcoma/Malignant Fibrous Histiocytoma; Brain Stem Glioma; Brain Tumor; Brain Tumor, Brain Stern Glioma; Brain Tumor, Cerebellar Astrocytoma; Brain Tumor, Cerebral Astrocytoma/Malignant Glioma; Brain Tumor, Ependymoma; Brain Tumor, Medulloblastoma; Brain Tumor, Supratentorial Primitive Neuroectodermal Tumors; Brain Tumor, Visual Pathway and Hypothalamic Glioma; Breast Cancer, Female; Breast Cancer, Male; Bronchial Adenomas/Carcinoids; Burkitt's Lymphoma; Carcinoid Tumor; Central Nervous System Lymphoma; Cerebellar Astrocytoma; Cerebral Astrocytoma/Malignant Glioma; Cervical Cancer; Chronic Lymphocytic Leukemia; Chronic Myelogenous Leukemia; Chronic Myeloproliferative Disorders; Myelodysplastic Syndromes; Colon Cancer; Colorectal Cancer; Cutaneous T-Cell Lymphoma; B-Cell Lymphoma Endometrial Cancer; Ependymoma; Esophageal Cancer; Esophageal Cancer, Ewing's Family of Tumors; Extracranial Germ Cell Tumor; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastrointestinal Carcinoid Tumor; Germ Cell Tumor, Extracranial; Germ Cell Tumor, Ovarian; Gestational Trophoblastic Tumor; Glioma; Glioma, Childhood Brain Stem; Glioma, Childhood Cerebral Astrocytoma; Glioma Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma; Hodgkin's Lymphoma During Pregnancy; Hypopharyngeal Cancer; Hypothalamic and Visual Pathway Glioma; Intraocular Melanoma; Islet Cell Carcinoma (Endocrine Pancreas); Kaposi's Sarcoma; Kidney (Renal Cell) Cancer; Kidney Cancer; Laryngeal Cancer; Leukemia, Acute Lymphoblastic; Leukemia, Acute Lymphoblastic; Leukemia, Acute Myeloid; Leukemia, Acute Myeloid; Leukemia, Chronic Lymphocytic; Leukemia; Chronic Myelogenous; Lip and Oral Cavity Cancer; Liver Cancer, Adult (Primary); Liver Cancer, Childhood (Primary); Lung Cancer, Non-Small Cell; Lung Cancer, Small Cell; Lymphoma, AIDS-Related; Lymphoma, Burkitt's; Lymphoma, Cutaneous T-Cell, see Mycosis Fungoides and Sezary Syndrome; Lymphoma, Hodgkin's; Lymphoma, Hodgkin's During Pregnancy; Lymphoma, Non-Hodgkin's; Lymphoma, Non-Hodgkin's During Pregnancy; Lymphoma, Primary Central Nervous System; Macroglobulinemia, Waldenstrom's; Malignant Fibrous Histiocytoma of Bone/Osteosarcoma; Medulloblastoma; Melanoma; Melanoma, Intraocular (Eye); Merkel Cell Carcinoma; Mesothelioma, Adult Malignant; Mesothelioma; Metastatic Squamous Neck Cancer with Occult Primary; Multiple Endocrine Neoplasia Syndrome; Multiple Myeloma/Plasma Cell Neoplasm' Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Neuroblastoma; Non-Hodgkin's Lymphoma; Non-Hodgkin's Lymphoma During Pregnancy; Oral Cancer; Oral Cavity Cancer, Lip and; Oropharyngeal Cancer; Osteosarcoma/Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer; Pancreatic Cancer, Islet Cell; Parathyroid Cancer; Penile Cancer; Pheochromocytoma; Pineoblastoma and Supratentorial Primitive Neuroectodermal Tumors; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Prostate Cancer; Rectal Cancer; Renal Cell (Kidney) Cancer; Renal Cell (Kidney) Cancer; Renal Pelvis and Ureter, Transitional Cell Cancer; Retinoblastoma; Rhabdomyosarcoma; Salivary Gland Cancer; Salivary Gland Cancer; Sarcoma, Ewing's Family of Tumors; Sarcoma, Kaposi's; Sarcoma, Soft Tissue; Sarcoma, Soft Tissue; Sarcoma, Uterine; Sezary Syndrome; Skin Cancer (non-Melanoma); Skin Cancer; Skin Cancer (Melanoma); Skin Carcinoma, Merkel Cell; Small Cell Lung Cancer; Small Intestine Cancer; Soft Tissue Sarcoma; Soft Tissue Sarcoma; Squamous Cell Carcinoma, see Skin Cancer (non-Melanoma); Squamous Neck Cancer with Occult Primary, Metastatic; Stomach (Gastric) Cancer; Stomach (Gastric) Cancer; Supratentorial Primitive Neuroectodermal Tumors; T-Cell Lymphoma, Cutaneous, see Mycosis Fungoides and Sezary Syndrome; Testicular Cancer; Thymoma; Thymoma and Thymic Carcinoma; Thyroid Cancer; Thyroid Cancer; Transitional Cell Cancer of the Renal Pelvis and Ureter; Trophoblastic Tumor, Gestational; Ureter and Renal Pelvis, Transitional Cell Cancer; Urethral Cancer; Uterine Cancer, Endometrial; Uterine Sarcoma; Vaginal Cancer; Visual Pathway and Hypothalamic Glioma; Vulvar Cancer; Waldenstrom's Macroglobulinemia; and Wilms' Tumor.

In one embodiment, the nucleolin-specific scFv is used to reduce cell viability of a cancer cell in a subject sample by 30 to 100% as compared to cells not exposed to a nucleolin-specific scFv. In one embodiment, a nucleolin-specific scFv is used to reduce cell viability of a cancer cell in a subject sample by 30 to 100% as compared cells not exposed to a nucleolin-specific scFv.

In one embodiment a nueleolin-specific scFv is administered to a human subject with one or more forms of cancer. In one embodiment a nucleolin-specific scFv is administered to a human subject with one or more forms of cancer. In one embodiment at least one of the forms of cancer is inhibited or killed by a nucleolin-specific scFv. In one embodiment an isolated nucleolin-specific scFv is administered to a human subject where the cancer is resistant to other cancer treatments. For example, cancers can be resistant to radiation therapy, chemotherapy, or biological therapy.

In one embodiment, a nucleolin-specific scFv is used to inhibit or kill a cell as part of an adjuvant therapy. In one embodiment, a nucleolin-specific scFv is used to inhibit or kill a cell as part of an adjuvant therapy. Adjuvant therapy as used herein refers to treatment given after the primary treatment to lower the risk that the cancer will come back.

In one embodiment, a nucleolin-specific scFv is used to inhibit or kill a cell of a non-malignant cell proliferative disorder wherein nucleolin is expressed on the cell surface or in the cytoplasm. For example, specific non-limiting examples of non-malignant cell proliferative disorders that can treated or inhibited with an anti-nucleolin antibody include but are not limited to warts, benign prostatic hyperplasia, skin tags, and non-malignant tumors. For example, a nucleolin-specific scFv can be used to determine such cell proliferative disorders as benign prostatic hyperplasia or unwanted genital warts by targeting the undesirable cells that characterize such conditions for removal. Expression of nucleolin on the cell surface of endothelial cells in tumors has been shown to be a unique marker of tumor angiogenesis. In one embodiment, a nucleolin-specific scFv is used to inhibit or kill in a subject a cell comprising an angiogenic tumor. An angiogenic tumor as used herein a tumor cell with a proliferation of a network of blood vessels that penetrate into cancerous growths, supplying nutrients and oxygen and removing waste products.

In one embodiment, a nucleolin-specific scFv is used to inhibit or kill in a subject a tumor cell under conditions of tumor hypoxia. Tumor hypoxia occurs in the situation where tumor cells have been deprived of oxygen. Tumor hypoxia can be a result of the high degree of cell proliferation undergone in tumor tissue, causing a higher cell density, and thus taxing the local oxygen supply.

In one embodiment, a nucleolin-specific scFv is used to inhibit or kill in subject a lymphocyte cell expressing human nucleolin on its surface. In one embodiment, the lymphocyte cell comprises a B cell, T cell, or natural killer cell. In one embodiment, the lymphocyte cell comprises a CD4-positive or CD8-positive cells.

In one embodiment, a nucleolin-specific scFv is used to inhibit or kill in a subject an activated lymphocyte or memory cell expressing human nucleolin on its surface. In a further embodiment, the activated lymphocyte comprises an activated B cell, T cell, or natural killer cell. In one embodiment, a human anti-nucleolin antibody is used to inhibit or kill a cell in a subject having an autoimmune disorder. In one embodiment, an isolated human anti-nucleolin monoclonal antibody is used to inhibit or kill a cell in a subject having an autoimmune disorder.

In one embodiment, a nucleolin-specific scFv is used to inhibit or kill a cell in a subject having an autoimmune disorder. CD40 and CD40 ligand are interactions mediate T-dependent B cell response and efficient T cell priming and nucleolin has been shown to interact with CD40 ligand. In one embodiment the cell is a lymphocyte. In one embodiment the lymphocyte is a B cell or T cell. In one embodiment the lymphocyte is activated. Exemplary autoimmune diseases or disorders which may be diagnosed with the use of a human anti-nucleolin antibody include, but are not limited to: alopecia areata, ankylosing spondylitis, antiphospholipid syndrome, autoimmune Addison's disease, asthma, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, diabetes, type 1 diabetes mellitus, diabetic retinopathy, eosinophilic fascites, fibromyalgia-fibromyositis, glomerulonephritis, Graves' disease, Guillain-Barre, Hashimoto's thyroiditis, Henoch-Schonlein purpura, idiopathic pulmonary fibrosis, idiopathic/autoimmune thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, pemphigus-related disorders (e.g., pemphigus vulgaris), pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjogren's syndrome, stiff-man syndrome, systemic lupus erythematosis (SLE), Sweet's syndrome, Still's disease, lupus erythematosus, takayasu arteritis, temporal arteristis/giant cell arteritis, ulcerative colitis, uveitis, vasculitides such as dermatitis herpetiformis vasculitis, vitiligo, and Wegener's granulomatosis. Examples of inflammatory disorders include, but are not limited to, asthma, encephilitis, inflammatory bowel disease, chronic obstructive pulmonary disease (COPD), allergic disorders, septic shock, pulmonary fibrosis, undifferentitated spondyloarthropathy, undifferentiated arthropathy, arthritis, inflammatory osteolysis, graft versus host disease, urticaria, Vogt-Koyanagi-Hareda syndrome, chronic inflammatory pneumonitis, and chronic inflammation resulting from chronic viral or bacterial infections.

In another embodiment, a nucleolin-specific scFv is used to inhibit or kill a cell in a subject infected by a virus. Examples of virus which can infect cells include but are not limited to: Retroviridae (e.g., human immunodeficiency viruses, such as HIV-1 (also referred to as HTLV-III, LAV or HTLV-III/LAV, or HIV-III); and other isolates, such as HIV-LP); Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronoviridae (e.g., coronaviruses); Rhabdoviradae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g. influenza viruses); Bungaviridae (e.g., Hantaan viruses, bunga viruses, phleboviruses and Nairo viruses); Arenaviridae (hemorrhagic fever viruses); Reoviridae (e.g., reoviruses, orbiviurses and rotaviruses); Bimaviridae; Hepadnaviridae (Hepatitis B virus); Parvovirida (parvoviruses), Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus); Rous sarcoma virus (RSV), avian leukemia virus (ALV), and avian myeloblastosis virus (AMV)) and C-type group B (including feline leukemia virus (FeLV), gibbon ape leukemia virus (GALV), spleen necrosis virus (SNV), reticuloendotheliosis virus (RV) and simian sarcoma virus (SSV)), D-type retroviruses include Mason-Pfizer monkey virus (MPMV) and simian retrovirus type 1 (SRV-1), the complex retroviruses including the subgroups of lentiviruses, T-cell leukemia viruses and the foamy viruses, lentiviruses including HIV-1, HIV-2, SIV, Visna virus, feline immunodeficiency virus (FIV), and equine infectious anemia virus (EIAV), simian T-cell leukemia virus (STLV), and bovine leukemia virus (BLV), the foamy viruses including human foamy virus (HEV), simian foamy virus (SFV) and bovine foamy virus (BFV), Poxviridae (variola viruses, vaccinia viruses, pox viruses); and Iridoviridae (e.g. African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses), Mycobacterium (Mycobacterium tuberculosis, M Bovis, M. avium-intracellulare, M leprae), Pneumococcus, Streptococcus, Staphylcococcus, Diphtheria, Listeria, Erysipelothrix, Anthrax, Tetanus, Clostridium, Mixed Anaerobes, Neisseria, Salmonella, Shigella, Hemophilus, Escherichia coli, Klebsiella, Enterobacter, Serratia, Pseudomonas, Bordatella, Francisella tularensis, Yersinia, Vibrio cholerae, Bartonella, Legionella, Spirochaetes (Treponema, Leptospira, Borrelia), Fungi, Actinomyces, Rickettsia, Mycoplasma, Chlamydia Protozoa (including Entamoeba, Plasmodium, Leishmania, Trypanosoma, Toxoplasma, Pneumocystis, Babasia, Giardia, Cryptosporidium, Trichomonas), Helminths (Trichinella, Wucheraria, Onchocerca, Schistosoma, Nematodes, Cestodes, Trematodes), and viral pneumonias. Additional examples of antigens which can be targets for compositions of the invention are known, such as those disclosed in U.S. Patent Publication No. 2007/0066554. In a further aspect of the invention, a conjugate can comprise an antigen or cellular component as described herein, but in addition to a targeting moiety and an immunostimulatory nucleic acid molecule.

In one embodiment, a nucleolin -specific scFv is used to inhibit or kill a cell in a sample from a subject as an indicator for the presence of a disease. Examples of diseases tested include but are not limited to malignant tumor, non-malignant tumor, cancer, autoimmune disease, inflammatory disease, and infectious disease.

The presently disclosed subject matter includes functional equivalents of the antibodies of the presently disclosed subject matter. As used herein, the phrase “functional equivalent” as it refers to an scFv means a molecule that has binding characteristics that are comparable to those of a given scFv. In some embodiments, chimerized, humanized, and human single chain antibodies, as well as fragments thereof, are considered functional equivalents of the corresponding antibodies upon which they are based.

Functional equivalents also include polypeptides with amino acid sequences substantially the same as the amino acid sequence of the variable or hypervariable regions of the antibodies of the presently disclosed subject matter. As used herein with respect to nucleic acid and/or amino acid sequences, the phrase “substantially the same” refers to a biosequence with in some embodiments at least 80%, in some embodiments at least 85%, in some embodiments at least about 90%, in some embodiments at least 91%, in some embodiments at least 92%, in some embodiments at least 93%, in some embodiments at least 94%, in some embodiments at least 95%, in some embodiments at least 96%, in some embodiments at least 97%, in some embodiments at least 98%, and in some embodiments at least about 99% sequence identity to another nucleic acid and/or amino acid sequence, as determined by the FASTA search method in accordance with Pearson & Lipman, 1988. In some embodiments, the percent identity calculation is performed over the full length of the nucleic acid and/or amino acid sequence of an antibody of the presently disclosed subject matter.

Engineering scFvs

scFvs can be engineered by methods known in the art. For example, an scFv library can be created, and scFvs selected from the library. For example, preferred amino acid residues can be substituted (or alternatively, amino acid residues to be excluded) at amino acid positions of interest (e.g., amino acid positions identified by comparing a database of scFv sequences having at least one desirable property, e.g., as selected with QC assay, versus a database of mature antibody sequences, e.g., the Kabat database) in an immunobinder. Disclosed herein are methods of identifying an amino acid position for mutation in a single chain antibody (scFv), the scFv having VH and VL amino acid sequences, the method comprising: a) entering the scFv VH, VL or VH and VL amino acid sequences into a database that comprises a multiplicity of antibody VH, VL or VH and VL amino acid sequences such that the scFv VH, VL or VH and VL amino acid sequences are aligned with the antibody VH, VL or VH and VL amino acid sequences of the database; b) comparing an amino acid position within the scFv VH or VL amino acid sequence with a corresponding position within the antibody VH or VL amino acid sequences of the database; c) determining whether the amino acid position within the scFv VH or VL amino acid sequence is occupied by an amino acid residue that is conserved at the corresponding position within the antibody VH or VL amino acid sequences of the database; and d) identifying the amino acid position within the scFv VH or VL amino acid sequence as an amino acid position for mutation when the amino acid position is occupied by an amino acid residue that is not conserved at the corresponding position within the antibody VH or VL amino acid sequences of the database.

Treatment Methods

Disclosed herein are compositions comprising an scFv and a pharmaceutically acceptable carrier. For example, disclosed are compositions useful for the treatment of cancer comprising a therapeutically effective amount of an scFv. For instance, the antibody fragment can be, directly or indirectly, associated with or linked to an effector moiety having therapeutic activity, and the composition is suitable for the treatment of cancer or infection. The effector moiety can be a radionuclide, therapeutic enzyme, anti-cancer drug, cytokine, cytotoxin, antibiotic, or anti-proliferative agent.

Disclosed herein is a method for in vivo treatment of a mammal having a NCL-expressing cancer comprising a step of administering to the mammal a therapeutically effective amount of a composition comprising an scFv.

Also disclosed is a method for suppressing tumor growth in a subject, the method comprising administering to a subject bearing a tumor an effective amount of an scFv composition, wherein the scFv is coupled to an anti-tumor composition. By “suppressing tumor growth” is meant that a tumor grows less than one which is not treated (a control). For example, suppressed tumor growth can mean that the tumor being treated grows 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100% less than the measured growth of a control over the same period of time.

The effector moiety disclosed herein can be a nucleic acid, such as microRNAs (miRNAs) or siRNAs. miRNAs are 21-23 nucleotide long RNAs that direct Argonaut proteins to bind to and repress complementary mRNA targets. The human genome contains more than 500 miRNAs, and each miRNA can repress hundreds of genes, regulating almost every cellular process. Individual miRNAs are often produced only in specific cell types or developmental stages. Inappropriate miRNA expression has been linked to a variety of diseases. For example, the let-7 miRNA prevents proliferation of cancer stem cells. miRNAs have roles in metabolic diseases such as obesity and diabetes; differentiation of adipocytes is promoted by miR-143, and insulin secretion is regulated by miR-375 in pancreatic-islet cells. Mutation of just a single nucleotide in the sequence of a miRNA or its mRNA target can eliminate target regulation. Mutation of the fifth nucleotide of miR-96 is associated with autosomal dominant, progressive, high-frequency hearing loss in humans; the mutation decreases the levels of miR-96 and impairs target mRNA repression. A different mutation in miR-96 was discovered in a mouse mutant with hair cell defects and progressive hearing loss. In contrast to mutation of miRNAs, normal miR-122 participates in the development of liver disease: hepatitis C virus (HCV) hijacks this miRNA, making miR-122 required for HCV to replicate in the liver. Some viruses express their own miRNAs, presumably to repress cellular mRNAs that would otherwise interfere with viral infection. Tissue-specific miRNAs may also be involved in the pathogenesis of cardiovascular, muscular and neurodegenerative diseases. Thus, molecules that alter the function or abundance of specific miRNAs represent a strategy for treating human disease.

In general, miRNA therapeutic approaches can be divided into two different categories: (1) miRNA inhibition therapy when the target miRNA is overexpressed and (2) miRNA replacement therapy when the miRNA is repressed. Therapeutic targeting of microRNAs can be accomplished either by direct inhibition or replacement of miRNAs or by targeting specific genes and therefore regulating the expression of specific miRNAs. For this purpose small-interfering RNAs (siRNAs) and genetically encoded expression vectors encoding small hairpin RNAs (shRNAs) are used.

The antibody fragment disclosed herein can be conjugated with an miRNA. For example, an scFv can comprise a stretch of positively charged amino acids. For instance, 4LB5 can comprise 6 histidines in a row. At a pH 7.0-8.0 (the pH of 4LB5 following its purification), these histidines are positively charged and they spontaneously associate with negatively charged oligonucleotides such as synthetic microRNAs (available for purchase from a commercially available source such as Ambion). The microRNA is incubated with the antibody fragment and can then be administered to a subject in need thereof. The binding of 4LB5 (cancer cell specific binding section of the molecule) and its internalization drives the consequent internalization of the microRNA. The use of microRNAs therapeutically is discussed in more detail in Broderick et al. (MicroRNA Therapeutics; Gene Therapy (2011) 18, 1104-1110), herein incorporated by reference in its entirety.

Administration

The scFvs of the invention may be administered to a mammal in accordance with the aforementioned methods of treatment in an amount sufficient to produce such effect to a therapeutic, prophylactic, or diagnostic effect. Such antibodies of the invention can be administered to such mammal in a conventional dosage form prepared by combining the antibody of the invention with a conventional pharmaceutically acceptable carrier or vehicle, diluent, and/or excipient according to known techniques to form a suspension, injectable solution, or other formulation. It will be recognized by one of skill in the art that the form and character of the pharmaceutically acceptable carrier or diluent is dictated by the amount of active ingredient with which it is to be combined, the route of administration and other well-known variables.

Pharmaceutically acceptable formulations may include, e.g., a suitable solvent, preservatives such as benzyl alcohol if desired, and a buffer. Useful solvent may include, e.g., water, aqueous alcohols, glycols, and phosphate and carbonate esters. Such aqueous solutions contain no more than 50% by volume of organic solvent. Suspension-type formulations may include a liquid suspending medium as a carrier, e.g., aqueous polyvinylpyrrolidone, inert oils such as vegetable oils or highly refined mineral oils, or aqueous cellulose ethers such as aqueous carboxymethylcellulose. A thickener such as gelatin or an alginate may also be present, one or more natural or synthetic surfactants or antifoam agents may be used, and one or more suspending agents such as sorbitol or another sugar may be employed therein. Such formations may contain one or more adjuvants.

The route of administration of the scFv of the invention may be oral, parenteral, by inhalation or topical. The term parenteral as used herein includes intravenous, intramuscular, subcutaneous, rectal, vaginal or intraperitoneal administration. The subcutaneous, intravenous and intramuscular forms of parenteral administration are generally preferred. The daily parenteral and oral dosage regimens for employing humanized antibodies of the invention prophylactically or therapeutically will generally be in the range of about 0.005 to 100, but preferably about 0.5 to 10, milligrams per kilogram body weight per day.

The scFv of the invention may also be administered by inhalation. By “inhalation” is meant intranasal and oral inhalation administration. Appropriate dosage forms for such administration, such as an aerosol formulation or a metered dose inhaler, may be prepared by conventional techniques. The preferred dosage amount of a compound of the invention to be employed is generally within the range of about 0.1 to 1000 milligrams, preferably about 10 to 100 milligrams/kilogram body weight.

The scFv of the invention may also be administered topically. By topical administration is meant non-systemic administration. This includes the administration of a humanized antibody (or humanized/human antibody fragment) formulation of the invention externally to the epidermis or to the buccal cavity, and instillation of such an antibody into the ear, eye, or nose, and wherever it does not significantly enter the bloodstream. By systemic administration is meant oral, intravenous, intraperitoneal, subcutaneous, and intramuscular administration. The amount of an antibody required for therapeutic, prophylactic, or diagnostic effect will, of course, vary with the antibody chosen, the nature and severity of the condition being treated and the animal undergoing treatment, and is ultimately at the discretion of the physician. A suitable topical dose of an antibody of the invention will generally be within the range of about 1 to 100 milligrams per kilogram body weight daily.

Formulations

While it is possible for an antibody fragment to be administered alone, it is preferable to present it as a pharmaceutical formulation. The active ingredient may comprise, for topical administration, from 0.001% to 10% w/w, e.g., from 1% to 2% by weight of the formulation, although it may comprise as much as 10% w/w but preferably not in excess of 5% w/w and more preferably from 0.1% to 1% w/w of the formulation. The topical formulations of the present invention, comprise an active ingredient together with one or more acceptable carrier(s) therefor and optionally any other therapeutic ingredients(s). The carrier(s) must be “acceptable” in the sense of being compatible with the other ingredients of the formulation and not deleterious to the recipient thereof.

Formulations suitable for topical administration include liquid or semi-liquid preparations suitable for penetration through the skin to the site of where treatment is required, such as liniments, lotions, creams, ointments or pastes, and drops suitable for administration to the eye, ear, or nose. Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified and sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogels. The formulation may incorporate any suitable surface active agent such as an anionic, cationic or non-ionic surface active such as sorbitan esters or polyoxyethylene derivatives thereof. Suspending agents such as natural gums, cellulose derivatives or inorganic materials such as silicaceous silicas, and other ingredients such as lanolin, may also be included.

Kits according to the present invention include scFvs as disclosed herein, and instructions for their use. Frozen or lyophilized human antibody fragments to be reconstituted, respectively, by thawing (optionally followed by further dilution) or by suspension in a (preferably buffered) liquid vehicle can also be used in these kits. The kits may also include buffer and/or excipient solutions (in liquid or frozen form)—or buffer and/or excipient powder preparations to be reconstituted with water—for the purpose of mixing with the humanized or human antibodies or human antibody fragments to produce a formulation suitable for administration. Thus, preferably the kits containing the humanized or human antibodies or human antibody fragments are frozen, lyophilized, pre-diluted, or pre-mixed at such a concentration that the addition of a predetermined amount of heat, of water, or of a solution provided in the kit will result in a formulation of sufficient concentration and pH as to be effective for in vivo or in vitro use in the treatment or diagnosis of cancer. Preferably, such a kit will also comprise instructions for reconstituting and using the humanized antibody or human antibody fragment composition to treat or detect cancer. The kit may also comprise two or more component parts for the reconstituted active composition. For example, a second component part—in addition to the humanized antibodies or human antibody fragments—may be bifunctional chelant, bifunctional chelate, or a therapeutic agent such as a radionuclide, which when mixed with the humanized antibodies or human antibody fragments forms a conjugated system therewith. The above-noted buffers, excipients, and other component parts can be sold separately or together with the kit.

It will be recognized by one of skill in the art that the optimal quantity and spacing of individual dosages of a humanized antibody or human antibody fragment of the invention will be determined by the nature and extent of the condition being treated, the form, route and site of administration, and the particular animal being treated, and that such optima can be determined by conventional techniques. It will also be appreciated by one of skill in the art that the optimal course of treatment, i.e., the number of doses of an antibody or fragment thereof of the invention given per day for a defined number of days, can be ascertained by those skilled in the art using conventional course of treatment determination tests.

Active Agents

The compositions of the presently disclosed subject matter can comprise an active agent, wherein the active agent comprises a therapeutic moiety, a diagnostic moiety, and/or a biologically active moiety. As used herein, the phrase “active agent” thus refers to a component of the presently disclosed compositions that provides a therapeutic benefit to a subject, permits visualization of cells or tissues in which the compositions of the presently disclosed subject matter accumulate, detection of epitopes to which the presently disclosed scFvs bind, and/or enhances any of these activities. In some embodiments, an active agent of the presently disclosed subject matter is selected from the group consisting of a radioactive molecule (including, but not limited to radionuclides and radioisotopes), a sensitizer molecule, an imaging agent or other detectable agent, a toxin, a cytotoxin, an anti-angiogenic agent, an anti-tumor agent, a chemotherapeutic agent, an immunomodulator, a cytokine, a reporter group, and combinations thereof it is understood that these categories are not intended to be mutually exclusive, as some radioactive molecules, for example, are also chemotherapeutic agents, some immunomodulators are cytokines, etc.

In some embodiments, an active agent comprises a chemotherapeutic. Various chemotherapeutics are known to one of ordinary skill in the art, and include, but are not limited to alkylating agents such as nitrogen mustards (e.g., Chlorambucil, Cyclophosphamide, Isofamide, Mechlorethamine, Melphalan, Uracil mustard), aziridines (e.g., Thiotepa), methanesulfonate esters (e.g., Busulfan), nitroso ureas (e.g., Carmustine, Lomustine, Streptozocin), platinum complexes (e.g., Cisplatin, Carboplatin), and bioreductive alkylators (e.g., Mitomycin C, Procarbazine); DNA strand breaking agents (e.g., Bleomycin); DNA topoisomerase I inhibitors (e.g., camptothecin and derivatives thereof including, but not limited to 10-hydroxycamptothecin), DNA topoisomerase II inhibitors (e.g., Amsacrine, Dactinomycin, Daunorubicin, Doxorubicin, Idarubicin, Mitoxantrone, Etoposide, Teniposide, Podophyllotoxin); DNA minor groove binders (e.g., Plicamycin); anti-metabolites such as folate antagonists (e.g., Methotrexate and trimetrexate), pyrimidine antagonists (e.g., Fluorouracil, Fluorodeoxyuridine, CB3717, Azacytidine, Cytarabine, Floxuridine), purine antagonists (e.g., Mercaptopurine, 6-Thioguanine, Fludarabine, Pentostatin), sugar modified analogs (e.g., Cyctrabine, Fludarabine), and ribonucleotide reductase inhibitors (e.g., Hydroxyurea); tubulin interactive agents (e.g., Vincristine, Vinblastine, Paclitaxel); adrenal corticosteroids (e.g., Prednisone, Dexamethasone, Methylprednisolone, Prednisolone); hormonal blocking agents such as estrogens and related compounds (e.g., Ethinyl Estradiol, Diethylstilbesterol, Chlorotrianisene, Idenestrol), progestins (e.g., Hydroxyprogesterone caproate, Medroxyprogesterone, Megestrol), androgens (e.g., Testosterone, Testosterone propionate; Fluoxymesterone, Methyltestosterone), leutinizing hormone releasing hormone agents and/or gonadotropin-releasing hormone antagonists (e.g., Leuprolide acetate; Goserelin acetate), anti-estrogenic agents (e.g., Tamoxifen), anti-androgen agents (e.g., Flutamide), and anti-adrenal agents (e.g., Mitotane, Aminoglutethimide). Other chemotherapeutics include, but are not limited to Taxol, retinoic acid and derivatives thereof (e.g., 13-cis-retinoic acid, all-trans-retinoic acid, and 9-cis-retinoic acid), sulfathiazole, mitomycin C, mycophenolic acid, sulfadiethoxane, and gemcitabine (4-amino-1-(2-deoxy-2,2-difluoro-D-eryi/7ro-pentofuranosyl)pyhmidin-2(1H)-on-2′,2′-difluoro-2-deoxycytidine).

The subject scFvs may also be administered in combination with other anti-cancer agents, e.g., other antibodies or drugs. Also, the subject human scFvs may be directly or indirectly attached to effector having therapeutic activity. Suitable effector moieties include by way of example cytokines (IL-2, TNF, interferons, colony stimulating factors, IL-1, etc.), cytotoxins (Pseudomonas exotoxin, ricin, abrin, etc.), radionuclides, such as 90Y, 131I, 99mTc, 111In, 125I, among others, drugs (methotrexate, daunorubicin, doxorubicin, etc.), immunomodulators, therapeutic enzymes (e.g., beta-galactosidase), anti-proliferative agents, etc. The attachment of antibodies to desired effectors is well known. See, e.g., U.S. Pat. No. 5,435,990 to Cheng et al. Moreover, bifunctional linkers for facilitating such attachment are well known and widely available. Also, chelators (chelants and chelates) providing for attachment of radionuclides are well known and available.

The compositions of the presently disclosed subject matter can further comprise a drug carrier to facilitate drug preparation and administration. Any suitable drug delivery vehicle or carrier can be used, including but not limited to a gene therapy vector (e.g., a viral vector or a plasmid), a microcapsule, for example a microsphere or a nanosphere (Manome et al., 1994; Hallahan et al., 2001 b; Saltzman & Fung, 1997), a peptide (U.S. Pat. Nos. 6,127,339 and 5,574,172), a glycosaminoglycan (U.S. Pat. No. 6,106,866), a fatty acid (U.S. Pat. No. 5,994,392), a fatty emulsion (U.S. Pat. No. 5,651,991), a lipid or lipid derivative (U.S. Pat. No. 5,786,387), collagen (U.S. Pat. No. 5,922,356), a polysaccharide or derivative thereof (U.S. Pat. No. 5,688,931), a nanosuspension (U.S. Pat. No. 5,858,410), a polymeric micelle or conjugate (Goldman et al., 1997; U.S. Pat. Nos. 4,551,482; 5,714,166; 5,510,103; 5,490,840; and 5,855,900), and a polysome (U.S. Pat. No. 5,922,545).

In one embodiment, a nucleolin-specific scFv is conjugated to an enzymatically active toxin or fragment thereof. Examples of enzymatically active toxins and fragments thereof include, but are not limited to, diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), pokeweed antiviral protein, Momordica charantia inhibitor, curcin, crotin, Sapaonaria ofticinalis inhibitor, gelonin, mitogellin, restrictocin, phenomycin, enomycin, calicheamicins or the tricothecenes.

Conjugates of the antibody and cytotoxic agent can be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026.

In one embodiment, a nucleolin-specific scFv is conjugated to a cytokine. The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin, vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1a, IL-2, IL-3, IL-4, IL-5, IL-6, IL-7, IL-8, IL-9, IL-11, IL-12, and other polypeptide factors including LIF and kit ligand (KL). As used herein, the term cytokine includes proteins from natural sources or from recombinant cell culture and biologically active equivalents of the native sequence cytokines.

In another embodiment, a nucleolin-specific scFv is conjugated to an anti-viral agent. Example of anti-viral agents that can be used with an isolated human anti-nucleolin antibody include, but are not limited to, substrates and substrate analogs, inhibitors and other agents that severely impair, debilitate or otherwise destroy virus-infected cells. Substrate analogs include amino acid and nucleoside analogs. Substrates can be conjugated with toxins or other viricidal substances. Inhibitors include integrase inhibitors, protease inhibitors, polymerase inhibitors and transcriptase inhibitors such as reverse transcriptase inhibitors.

Specific antiviral agents that can be used with a nucleolin-specific scFv include, but are not limited to, ganciclovir, valganciclovir, oseltamivir (Tamiflu), zanamivir (Relenza), abacavir, aciclovir, acyclovir, adefovir, amantadine, amprenavir, ampligen, arbidol, atazanavir, atripla, boceprevir, cidofovir, combivir, darunavir, delavirdine, didanosine, docosanol, edoxudine, efavirenz, emtricitabine, enfuvirtide, entecavir, famciclovir, fomivirsen, fosamprenavir, foscarnet, fosfonet, fusion inhibitors (e.g., enfuvirtide), ibacitabine, immunovir, idoxuridine, imiquimod, indinavir, inosine, integrase inhibitor, interferon type III, interferon type II, interferon type I, interferon, lamivudine, lopinavir, loviride, maraviroc, moroxydine, nelfinavir, nevirapine, nexavir, nucleoside analogues, peginterferon alfa-2a, penciclovir, peramivir, pleconaril, podophyllotoxin, protease inhibitor, raltegravir, reverse transcriptase inhibitor, ribavirin, rimantadine, ritonavir, pyrimidine antiviral, saquinavir, stavudine, synergistic enhancer (antiretroviral), tenofovir, tenofovir disoproxil, tipranavir, trifluridine, trizivir, tromantadine, truvada, valaciclovir (Valtrex), victiviroc, vidarabine, viramidine, zalcitabine, and zidovudine.

Examples of nucleoside analogs that can be used with a nucleolin-specific scFv include acyclovir (ACV), ganciclovir (GCV), famciclovir, foscarnet, ribavirin, zalcitabine (ddC), zidovudine (AZT), stavudine (D4T), lamivudine (3TC), didanosine (ddI), cytarabine, dideoxyadenosine, edoxudine, floxuridine, idozuridine, inosine pranobex, 2′-deoxy-5-(methylamino)uridine, trifluridine and vidarabine.

The scFvs disclosed herein can also be conjugated with active enyzmes, such as RNAses. Furthermore, PEGylation or discrete PEGylation can be used to increase the in vivo half life of scFvs, or to affect the biodistribution, pharmacokinetic, and pharmacodynamic properties of the scFv.

Detection Methods

Disclosed are compositions suitable for the in vivo or in vitro detection of cancer comprising a diagnostically effective amount of an scFv disclosed herein. The scFv can be, directly or indirectly, associated with or linked to a detectable label, and the composition can be suitable for detection of cancer. Also disclosed is a method for in vitro immunodetection of Nucleolin-expressing cancer cells comprising a step of contacting the cancer cells with a composition comprising an scFv of the present invention. The scFv can be bound to a solid support, for example.

Also disclosed is a method of in vivo immunodetection of NCL-expressing cancer cells in a mammal comprising a step of administering to the mammal a diagnostically effective amount of a composition comprising the scFv of the present invention.

For diagnostic applications, a detectable amount of a composition of the presently disclosed subject matter is administered to a subject. A “detectable amount”, as used herein to refer to a composition, refers to a dose of such a composition that the presence of the composition can be determined in vivo or in vitro. A detectable amount will vary according to a variety of factors, including but not limited to chemical features of the composition being labeled, the detectable label, the labeling methods, the method of imaging and parameters related thereto, metabolism of the labeled drug in the subject, the stability of the label (including, but not limited to the half-life of a radionuclide label), the time elapsed following administration of the composition prior to imaging, the route of administration, the physical condition and prior medical history of the subject, and the size and longevity of the tumor or suspected tumor. Thus, a detectable amount can vary and can be tailored to a particular application. After study of the present disclosure, it is within the skill of one in the art to determine such a detectable amount.

As used herein, the terms “detectable moiety”, “detectable label”, and “detectable agent” refer to any molecule that can be detected by any moiety that can be added to an antibody fragment that allows for the detection of the antibody fragment in vitro and/or in vivo. Representative detectable moieties include, but are not limited to, chromophores, fluorescent moieties, enzymes, antigens, groups with specific reactivity, chemiluminescent moieties, and electrochemically detectable moieties, etc. In some embodiments, the antibodies are biotinylated.

Detection and imaging of the antibody fragment is tunable, such that imaging can be performed in under 1, 2, 4, 6, 12, or 18, 24, 36, or 48 hours, or any amount below, above, or between this amount. It has been demonstrated that PEGs/larger fragments increase serum half-life by 2, 3, 4, 5, 6, 7, 8, 9, 10, 15, 20, 25, 30, 35, 40, 45, 50, 55, 60, 65, 70, 75, 80, 85, 90, 95, or 100%, or 2, 3, 4, 5, 6, 7, 8, 9, or 10 or more times compared to a smaller fragment. This allows for imaging at different time points. For therapeutic purposes, it allows for an increase in the therapeutic window.

Detectable Moieties

In some embodiments, a detectable moiety comprises a fluorophore. Any fluorophore can be employed with the compositions of the presently disclosed subject matter, provided that the conjugation of fluorophore results in a composition that is detectable either in vivo (e.g., after administration to a subject) and/or in vitro, and further does not negatively impact the ability of the antibody fragment to bind to its epitope. Representative fluorophores include, but are not limited to 7-dimethylaminocoumarin-3-carboxylic acid, dansyl chloride, nitrobenzodiazolamine (NBD), dabsyl chloride, cinnamic acid, fluorescein carboxylic acid, Nile Blue, tetramethylcarboxyrhodamine, tetraethylsulfohodamine, 5-carboxy-X-rhodamine (5-ROX), and 6-carboxy-X-rhodamine (6-ROX). It is understood that these representative fluorophores are exemplary only, and additional fluorophores can also be employed. For example, there the ALEXA FLUOR® dye series includes at least 19 different dyes that are characterized by different emission spectra. These dyes include ALEXA FLUOR® 350, 405, 430, 488, 500, 514, 532, 546, 555, 568, 594, 610, 633, 635, 647, 660, 680, 700, and 750 (available from Invitrogen Corp., Carlsbad, Calif., United States of America), and the choice of which dye to employ can be made by the skilled artisan after consideration of the instant specification based on criteria including, but not limited to the chemical compositions of the specific ALEXA FLUOR®, whether multiple detectable moieties are to be employed and the emission spectra of each, the detection technique to be employed, etc.

In some embodiments, a detectable moiety comprises a cyanine dye. Non-limiting examples of cyanine dyes that can be conjugated to the antibody fragments of the presently disclosed subject matter include the succinimide esters Cy5, Cy5.5, and Cy7, supplied by Amersham Biosciences (Piscataway, N.J., United States of America).

In some embodiments, a detectable moiety comprises a near infrared (NIR) dye. Non-limiting examples of near infrared dyes that can be conjugated to the scFv of the presently disclosed subject matter include NIR641, NIR664, NIT7000, and NIT782.

In some embodiments, the biotinylated scFvs are detected using a secondary antibody that comprises an avidin or streptavidin group and is also conjugated to a fluorescent label including, but not limited to Cy3, Cy5, Cy7, and any of the ALEXA FLUOR®® series of fluorescent labels available from INVITROGEN™ (Carlsbad, Calif., United States of America). In some embodiments, the scFv is directly labeled with a fluorescent label and cells that bind to the antibody fragment are separated by fluorescence-activated cell sorting. Additional detection strategies are known to the skilled artisan.

For diagnostic applications (including but not limited to detection applications and imaging applications), the antibodies of the presently disclosed subject matter can be labeled with a detectable moiety. The detectable moiety can be any one that is capable of producing, either directly or indirectly, a detectable signal. For example, a detectable moiety can be a radioisotope, such as but not limited to 3H, 14C, 32P, 35S, 125I, or 3 l; a fluorescent or chemiluminescent compound such as but not limited to fluorescein isothiocyanate, rhodamine, or luciferin; or an enzyme, such as but not limited to alkaline phosphatase, β-galactosidase, or horseradish peroxidase.

The presently disclosed subject matter further provides methods for diagnosing a tumor, wherein a tumor sample or biopsy is evaluated in vitro. In some embodiments, a targeting ligand of the presently disclosed subject matter comprises a detectable label such as a fluorescent label, an epitope tag, or a radioactive label, each described briefly herein below.

Detection of an Epitope Tag

If an epitope label has been used, a protein or compound that binds the epitope can be used to detect the epitope. A representative epitope label is biotin, which can be detected by binding of an avidin-conjugated fluorophore, for example avidin-FITC. Alternatively, the label can be detected by binding of an avidin-horseradish peroxidase (HRP) streptavidin conjugate, followed by colorimetric detection of an HRP enzymatic product. The production of a colorimetric or luminescent product/conjugate is measurable using a spectrophotometer or luminometer, respectively.

Autoradiographic Detection

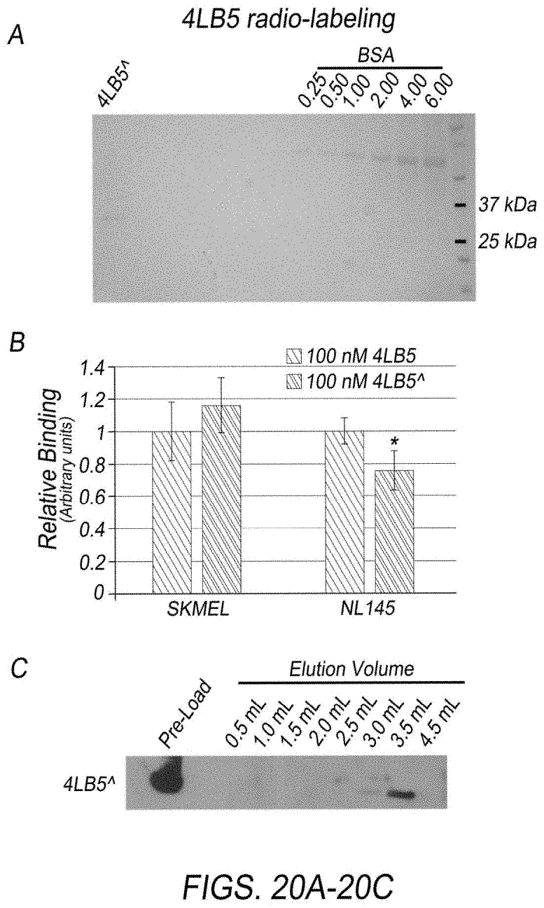

In the case of a radioactive label (e.g., ¹³¹I or ^{99m}Tc) detection can be accomplished by conventional autoradiography or by using a phosphorimager as is known to one of skill in the art. A preferred autoradiographic method employs photostimulable luminescence imaging plates (Fuji Medical Systems of Stamford, Conn., United States of America). Briefly, photostimulable luminescence is the quantity of light emitted from irradiated phosphorous plates following stimulation with a laser during scanning. The luminescent response of the plates is linearly proportional to the activity. This can be seen in FIG. 20.

Any method known in the art for conjugating an antibody to a detectable moiety can be employed.

Immunohistochemistry

Disclosed herein are methods of using immunohistochemistry (IHC) utilizing the scFvs disclosed herein to detect cancer. IHC detects target molecules through antigen-antibody complexes in a pathological specimen using enzyme-linked antigens or antibodies. The presence of the target molecule can then detected via an enzyme immunoassay.

A multitude of benefits are realized with IHC versus traditional immunofluorescence. For example, unlike immunofluorescence, IHC can be used with commonly used formalin-fixed paraffin-embedded tissue specimens. Pathological specimens, including histological tissue sections and/or other biological preparations such as tissue culture cells and PAP smears, are commonly used in diagnostic pathology and can be easily screened via IHC. Further, IHC staining is permanent and preserves cell morphology. A comparison of the cell morphology and antigen proliferation on two different slides can be useful in monitoring the progression of a disease.

Once a labeled antibody has been attached, either directly or indirectly, to the specimen, a substrate, specific for the enzyme, is added to the specimen. When the substrate is added, the enzyme label converts the substrate causing a color change that can be seen with light microscopy. The presence of a color change indicates the presence of the target molecule and allows an observer to determine, assess, and diagnose the disease level and severity.

In Vivo Imaging

The scFvs of the presently disclosed subject matter also are useful for in vivo imaging, wherein an antibody labeled with a detectable moiety such as a radio-opaque agent and/or a radioisotope is administered to a subject, in some embodiments via intravenous administration, and the presence and location of the labeled antibody in the host is assayed. This imaging technique can be useful in the staging and treatment of malignancies. This can be seen in FIG. 20.

Therefore, disclosed is a method of in vivo treatment of cancer comprising the steps of: (a) intravenously administering a radionuclide-labeled scFv; (b) thereafter detecting tumor cells using a radionuclide activity probe; and (c) thereafter removing the detected tumor cells by surgical excision.

Thus, in some embodiments, a composition of the presently disclosed subject matter comprises a label that can be detected in vivo. The term “in vivo” as used herein to describe imaging or detection methods, refers to generally non-invasive methods such as scintigraphic methods, magnetic resonance imaging, ultrasound, or fluorescence, each described briefly herein below. The term “non-invasive methods” does not exclude methods employing administration of a contrast agent to facilitate in vivo imaging.

In some embodiments, the detectable moiety can be conjugated or otherwise associated with the scFv of the presently disclosed subject matter, a therapeutic, a diagnostic agent, a drug carrier, or combinations thereof as set forth in more detail hereinabove. Following administration of the labeled composition to a subject, and after a time sufficient for binding, the biodistribution of the composition can be visualized. The term “time sufficient for binding” refers to a temporal duration that permits binding of the labeled agent to a radiation-induced target molecule.

Scintigraphic Imaging

Scintigraphic imaging methods include SPECT (Single Photon Emission Computed Tomography), PET (Positron Emission Tomography), gamma camera imaging, and rectilinear scanning. A gamma camera and a rectilinear scanner each represent instruments that detect radioactivity in a single plane. Most SPECT systems are based on the use of one or more gamma cameras that are rotated about the subject of analysis, and thus integrate radioactivity in more than one dimension. PET systems comprise an array of detectors in a ring that also detect radioactivity in multiple dimensions.

Imaging instruments suitable for practicing the detection and/or imaging methods of the presently disclosed subject matter, and instruction for using the same, are readily available from commercial sources. For example, a SPECT scanner can be used with a CT scanner, with coregistration of images. As in PET/CT, this allows location of tumors or tissues which may be seen on SPECT scintigraphy, but are difficult to precisely locate with regard to other anatomical structures. Both PET and SPECT systems are offered by ADAC of Milpitas, Calif., United States of America, and Siemens of Hoffman Estates, Illinois, United States of America. Related devices for scintigraphic imaging can also be used, such as a radio-imaging device that includes a plurality of sensors with collimating structures having a common source focus.

When scintigraphic imaging is employed, the detectable label comprises in some embodiments a radionuclide label, in some embodiments a radionuclide label selected from the group consisting of ¹⁸F, ⁶⁴Cu, ⁶⁵Cu, ⁶⁷Ga, ⁶⁸Ga, ⁷⁷Br, ^{80m}Br, ⁹⁵Ru, ⁹⁷Ru, ¹⁰³Ru, ¹⁰⁵Ru, ^{99m}Tc, ¹⁰⁷Hg, ²⁰³Hg, ¹²³I, ¹²⁴I, ¹²⁵I, ^{I 131}I, ¹³³I, ¹¹¹In, ^{99m}Re, ¹⁰⁵Re, ¹⁰¹Re, ¹⁸⁶Re; ¹⁸⁸Re, ^{121m}Te, ^{122m}Te, ^{125m}Te, ¹⁶⁵Tm, ¹⁶⁷Tm, ¹⁶⁸Tm, and nitride or oxide forms derived there from. In some embodiments, the radionuclide label comprises ¹³¹I or ^{99m}Tc.

Methods for radionuclide labeling of a molecule so as to be used in accordance with the disclosed methods are known in the art. For example, a targeting molecule can be derivatized so that a radioisotope can be bound directly to it. Alternatively, a linker can be added that to enable conjugation. Representative linkers include diethylenetriamine pentaacetate (DTPA)-isothiocyanate, succinimidyl 6-hydrazinium nicotinate hydrochloride (SHNH), and hexamethylpropylene amine oxime (U.S. Pat. No. 6,024,938). Additional methods can be found in U.S. Pat. No. 6,080,384.

When the labeling moiety is a radionuclide, stabilizers to prevent or minimize radiolytic damage, such as ascorbic acid, gentisic acid, or other appropriate antioxidants, can be added to the composition comprising the labeled targeting molecule.

Magnetic Resonance Imaging (MRI)

Magnetic resonance image-based techniques create images based on the relative relaxation rates of water protons in unique chemical environments. As used herein, the term “magnetic resonance imaging” refers to magnetic source techniques including convention magnetic resonance imaging, magnetization transfer imaging (MTI), proton magnetic resonance spectroscopy (MRS), diffusion-weighted imaging (DWI) and functional MR imaging.

Contrast agents for magnetic source imaging include but are not limited to paramagnetic or superparamagnetic ions, iron oxide particles, and water-soluble contrast agents. Paramagnetic and superparamagnetic ions can be selected from the group of metals including iron, copper, manganese, chromium, erbium, europium, dysprosium, holmium and gadolinium. Preferred metals are iron, manganese and gadolinium; most preferred is gadolinium.

Those skilled in the art of diagnostic labeling recognize that metal ions can be bound by chelating moieties, which in turn can be conjugated to a therapeutic agent in accordance with the methods of the presently disclosed subject matter. For example, gadolinium ions are chelated by diethylenetriaminepentaacetic acid (DTPA). Lanthanide ions are chelated by tetraazacyclododocane compounds. See U.S. Pat. Nos. 5,738,837 and 5,707,605. Alternatively, a contrast agent can be carried in a liposome.

Images derived used a magnetic source can be acquired using, for example, a superconducting quantum interference device magnetometer (SQUID, available with instruction from Quantum Design of San Diego, Calif., United States of America; see also U.S. Pat. No. 5,738,837).

Ultrasound

Ultrasound imaging can be used to obtain quantitative and structural information of a target tissue, including a tumor. Administration of a contrast agent, such as gas microbubbles, can enhance visualization of the target tissue during an ultrasound examination. In some embodiments, the contrast agent can be selectively targeted to the target tissue of interest, for example by using a peptide for guided drug delivery (e.g., radiation guided drug delivery) as disclosed herein. Representative agents for providing microbubbles in vivo include but are not limited to gas-filled lipophilic or lipid—based bubbles U.S. Pat. Nos. 6,245,318; 6,231,834; 6,221,018; and 5,088,499). In addition, gas or liquid can be entrapped in porous inorganic particles that facilitate microbubble release upon delivery to a subject (U.S. Pat. Nos. 6,254,852 and 5,147,631).

Gases, liquids, and combinations thereof suitable for use with the presently disclosed subject matter include air; nitrogen; oxygen; is carbon dioxide; hydrogen; nitrous oxide; an inert gas such as helium, argon, xenon or krypton; a sulfur fluoride such as sulfur hexafluoride, disulfur decafluoride or trifluoromethylsulfur pentafluoride; selenium hexafluoride, an optionally halogenated silane such as tetramethylsilane, a low molecular weight hydrocarbon (e.g. containing up to 7 carbon atoms), for example an alkane such as methane, ethane, a propane, a butane or a pentane, a cycloalkane such as cyclobutane or cyclopentane, an alkene such as propene or a butene, or an alkyne such as acetylene; an ether; a ketone; an ester; a halogenated low molecular weight hydrocarbon (e.g. containing up to 7 carbon atoms); or a mixture of any of the foregoing. Halogenated hydrocarbon gases can show extended longevity, and thus are preferred for some applications. Representative gases of this group include decafluorobutane, octafluorocyclobutane, decafluoroisobutane, octafluoropropane, octafluorocyclopropane dodecafluoropentane decafluorocyclopentane, decafluoroisopentane, perfluoropexane, perfluorocyclohexane, perfluoroisohexane, sulfur hexafluoride, and perfluorooctaines, perfluorononanes; perfluorodecanes, optionally brominated.

Attachment of targeting ligands to lipophilic bubbles can be accomplished via chemical crosslinking agents in accordance with standard protein-polymer or protein-lipid attachment methods (e.g., via carbodiimide (EDC) or thiopropionate (SPDP)). To improve targeting efficiency, large gas-filled bubbles can be coupled to a targeting ligand using a flexible spacer arm, such as a branched or linear synthetic polymer (U.S. Pat. No. 6,245,318). A targeting ligand can be attached to the porous inorganic particles by coating, adsorbing, layering, or reacting the outside surface of the particle with the targeting ligand (U.S. Pat. No. 6,254,852).

Fluorescence Imaging

Non-invasive imaging methods can also comprise detection of a fluorescent label. A drug comprising a lipophilic component (therapeutic agent, diagnostic agent, vector, or drug carrier) can be labeled with any one of a variety of lipophilic dyes that are suitable for in vivo imaging. Representative labels include but are not limited to carbocyanine and aminostyryl dyes, preferably long chain dialkyl carbocyanines (e.g., Dil, DiO, and DiD available from Molecular Probes Inc. of Eugene, Oreg., United States of America) and dialkylaminostyryl dyes. Lipophilic fluorescent labels can be incorporated using methods known to one of skill in the art. For example VYBRANT™ cell labeling solutions are effective for labeling of cultured cells of other lipophilic components (Molecular Probes Inc. of Eugene, Oreg., United States of America).

A fluorescent label can also comprise sulfonated cyanine dyes, including Cy5.5 and Cy5 (available from Amersham of Arlington Heights, Ill., United States of America). IRD41 and IRD700 (available from Li-Cor, Inc. of Lincoln, Nebr.), NIR-1 (available from Dejindo of Kumamoto, Japan), and LaJolla Blue.

In addition, a fluorescent label can comprise an organic chelate derived from lanthanide ions, for example fluorescent chelates of terbium and europium (U.S. Pat. No. 5,928,627). Such labels can be conjugated or covalently linked to a drug as disclosed therein.

For in vivo detection of a fluorescent label, an image is created using emission and absorbance spectra that are appropriate for the particular label used. The image can be visualized, for example, by diffuse optical spectroscopy. Additional methods and imaging systems are described in U.S. Pat. Nos. 5,865,754; 6,083,486; and 6,246,901, among other places.

Radioimmunoguided System® (RIGS)

Another preferred application of the scFvs is in the Radioimmunoguided System®. This technique, also known as the RIGS® System involves the intravenous administration of a radiolabeled monoclonal antibody or its fragment prior to surgery. After allowing for tumor uptake and blood clearance of radioactivity, the patient is taken to the operating room where surgical exploration is effected with the aid of a hand-held gamma activity probe, e.g., Neoprobe®1000. This helps the surgeon identify the tumor metastases and improve the complications of excision. The RIGS® system is advantageous because it allows for the detection of tumors not otherwise detectable by visual inspection and/or palpation. See, O'Dwyer et al, Arch. Surg., 121:1 391-1394 (1986). This technique is described in detail in Hinkle et al, Antibody, Immunoconjugates and Radiopharmacouticals, 4:(3)339-358 (1991) (citing numerous references describing this technique). This reference also discloses the use of this technique with the CC49 monoclonal antibody itself. This technique is particularly useful for cancers of the colon, breast, pancreas, and ovaries.

In some embodiments, the scFvs of the presently disclosed subject matter are employed for in vivo imaging of tumors, wherein a composition of the presently disclosed subject matter that has been labeled with an imaging moiety such as a radio-opaque agent, a radioisotope, or other imaging agent is administered to a subject, and the presence and location of the detectibly-labeled composition in the subject is assayed. This imaging technique can be useful in the staging and treatment of malignancies. In some embodiments, an antibody is labeled with any moiety that is detectable in situ in a subject, for example by nuclear magnetic resonance, radiology, or other detection methods known in the art.

As such, the presently disclosed subject matter also provides methods for detecting tumors in subjects. In some embodiments, the presently disclosed methods comprise (a) administering to the subject a composition comprising the scFv of the presently disclosed subject matter conjugated to a detectable label; and (b) detecting the detectable label to thereby detect the tumor.

Methods for Predicting the Recurrence and/or Progression of Cancer in a Subject

In some embodiments, the presently disclosed subject matter also provides methods for predicting the recurrence of cancer in a subject. In some embodiments, the methods comprise (a) isolating a biological sample comprising cells from a subject with a cancer; (b) contacting the biological sample with scFv of the presently disclosed subject matter; and (c) identifying in the biological sample one or more cells that bind to the scFv of the presently disclosed subject matter, whereby the recurrence of a cancer is predicted in the subject. With respect to these methods, the identification of cells that bind to the scFvs of the presently disclosed subject matter can be indicative of a recurrence of a subject's cancer when the subject had previously been negative for such circulating cells. In some embodiments, the presence of cells that bind to the one or more of the antibody fragments of the presently disclosed subject matter indicates that the subject is at enhanced risk of metastatic disease relative to a subject that is negative for such cells.

Methods for Prognosing Progression of Cancer

The presently disclosed subject matter also provides methods for prognosing progression of a cancer in subjects. In some embodiments, the methods comprise isolating a biological sample comprising cells from a subject with a cancer; contacting the biological sample with the scFv of the presently disclosed subject matter under conditions sufficient for the scFv to bind to an epitope present on a tumor and/or a cancer cell, if present, in the biological sample; and identifying in the biological sample one or more cells that bind to the scFv, whereby progression of a cancer is prognosed in the subject. In some embodiments, the biological sample comprises a blood sample, a lymph sample, or a fraction thereof. In some embodiments, the cancer is an adenocarcinoma or colon cancer.

As used herein, the phrase “prognosing progression of a cancer” refers to evaluating indicia of a cancer disease at a given time point and comparing the same to the indicia of the cancer disease taken at an earlier time point, wherein the comparison is indicative of a progression of the cancer in the subject. In some embodiments, progression of the cancer comprises metastasis of the cancer in the subject.

Other Uses

The antibodies of the presently disclosed subject matter can also be employed in various assay methods, such as but not limited to competitive binding assays, direct and indirect sandwich assays, and immunoprecipitation assays.

The antibodies of the presently disclosed subject matter also are useful as affinity purification agents. In this process, one or more antibodies are immobilized on a suitable support (such as, but not limited to a Sephadex resin or filter paper) using methods well known in the art. See e.g., Harlow & Lane, 1988.

Making scFvs

Also disclosed are methods of making scFvs comprising: (a) culturing an isolated cell comprising a vector comprising a nucleic acid sequence encoding an scFv as disclosed herein, under conditions such that said scFv is expressed; and (b) recovering said scFv from the cell.

As disclosed herein, the scFvs disclosed herein can be made by a variety of methods. Importantly, a VH and VL domain are present, and they are linked together.

Having generally described the invention, the same will be more readily understood by reference to the following examples, which are provided by way of illustration and are not intended as limiting.

Without further description, it is believed that one of ordinary skill in the art can, using the preceding description and the following illustrative examples, make and utilize the alterations detected in the present invention and practice the claimed methods. The following working examples therefore, specifically point out preferred embodiments of the present invention, and are not to be construed as limiting in any way the remainder of the disclosure.

### EXAMPLES

### Example 1

### A Human Anti-Nucleolin Recombinant Immunoagent for Cancer Therapy

Selection, Purification and Characterization of Anti-NCL scFv.

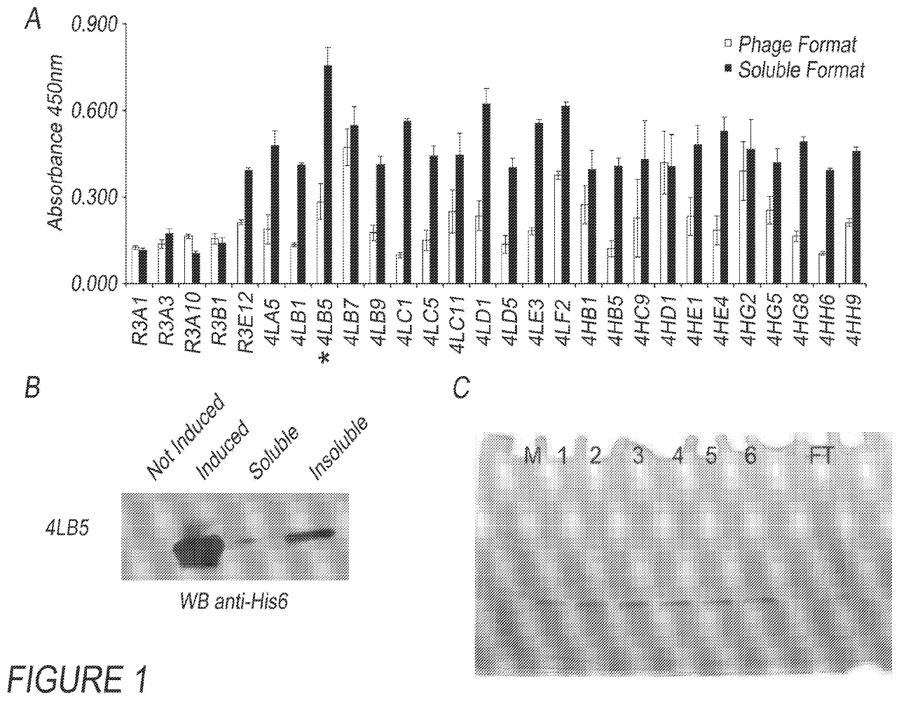

Using purified recombinant NCL-RBD (20 μg/ml) as bait, four rounds of selection of scFvs from the Griffin.1 library was performed (Marks J D, et al. (1991) JMB 222(3):581-597). A fifth round of selection, using a lower amount (10 μg/ml) of recombinant protein, was also carried out in order to select phages with higher affinity for NCL-RBD. Ninety-six clones (from the third, fourth and fifth round of selection) were analyzed by ELISA to select the best binders for recombinant NCL-RBD. Five, eleven and twelve phage clones were selected for further analysis from each round of selection, respectively. Results confirmed the binding of selected phages to recombinant NCL-RBD (FIG. 1A).

Selected anti-NCL scFvs clones were then analyzed in their soluble form by transforming bacterial SF110 cells with the pHEN2 phagemid vector (Nissim A, et al. (1994) The EMBO journal 13(3):692-698) extracted from these clones. Periplasmic extracts, obtained following isopropyl-1-thio-β-D-galactopyranoside induction, were analyzed by ELISA (FIG. 1A), and clone 4LB5, identified in the fifth round of selection, exhibited the greatest affinity in the soluble form. Sequence analysis of clone 4LB5 indicated that the scFv VH belongs to the VH4 family (derived from the VH germ-line gene DP-71), whereas the VL belongs to the VL3 family (derived from the VL germ-line gene DPL-16).

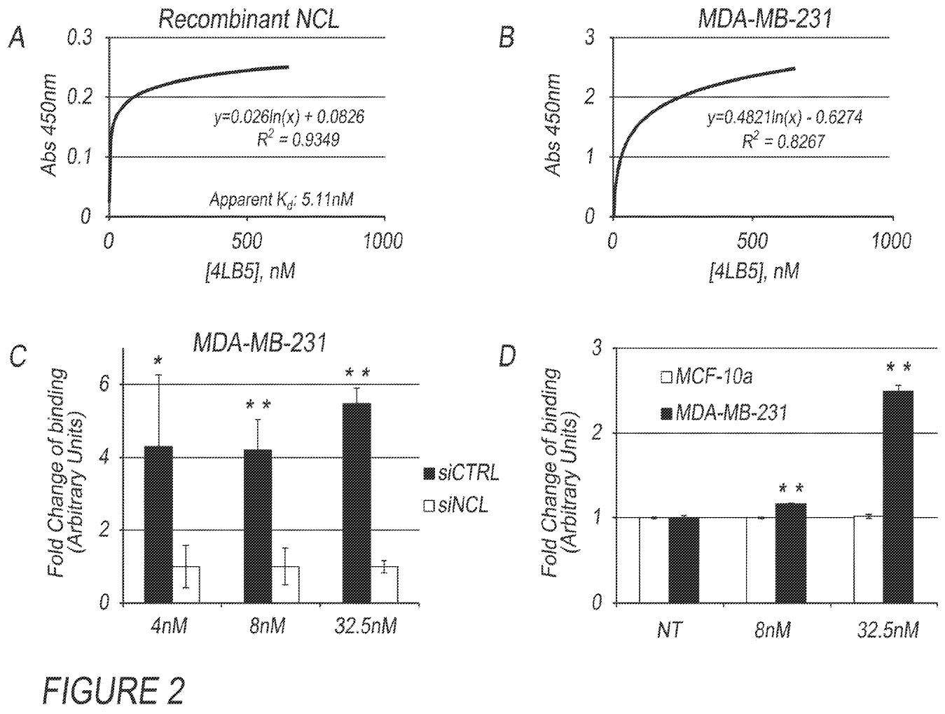

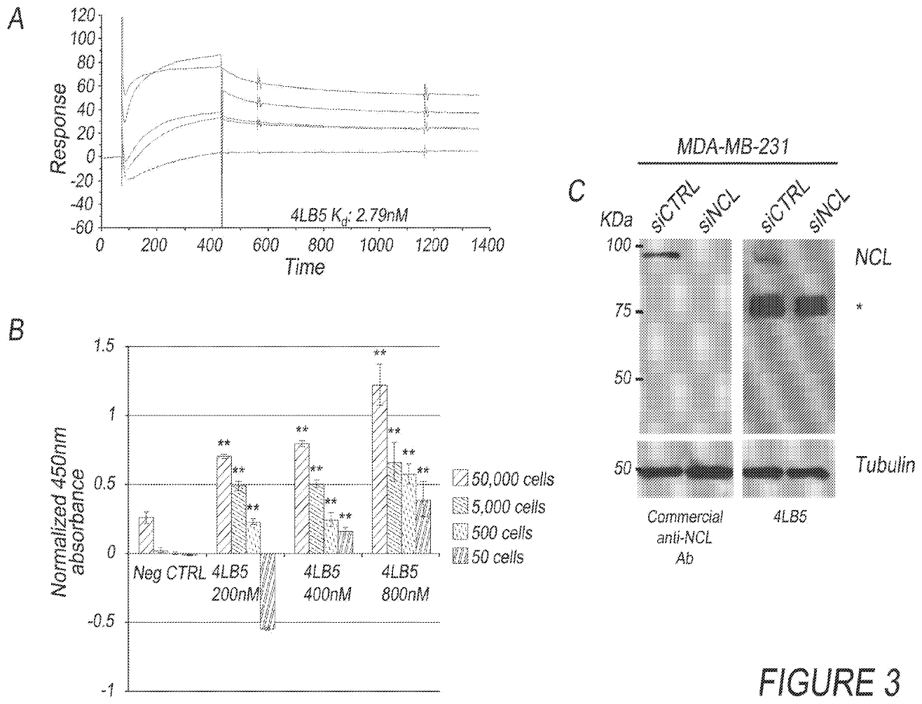

To obtain higher amounts of scFv, 4LB5 cDNA was subcloned into the pET22b(+) prokaryotic expression vector, fused with a C-terminal hexahistidine (His6) tag for its purification, and transformed in E. coli BL21(DE3) strain, However, fractionation of soluble and insoluble bacterial proteins revealed that 4LB5 scFv was mainly expressed in the insoluble form (FIG. 1B). For this reason, 4LB5 required denaturation prior to purification. SDS-PAGE analysis showed purified 4LB5 scFv to be approximately 27 kDa in size and about 90% pure (FIG. 1C). To determine its binding affinity to NCL-RBD, ELISA was performed using increasing concentrations (0.1-600 nM) of purified 4LB5. As shown in FIG. 2A, significant binding to NCL-RBD was observed, with an apparent K_{d} of 5.11 nM. To further quantify the binding properties of 4LB5, Surface Plasmon Resonance (SPR) analysis was performed with different concentrations of 4LB5 (FIG. 3A). In the experimental settings, 4LB5 displayed a dissociation rate constant (k_{d}) of 3.25×10⁻⁴s⁻¹, with equilibrium K_{D} value of 2.79 nM.

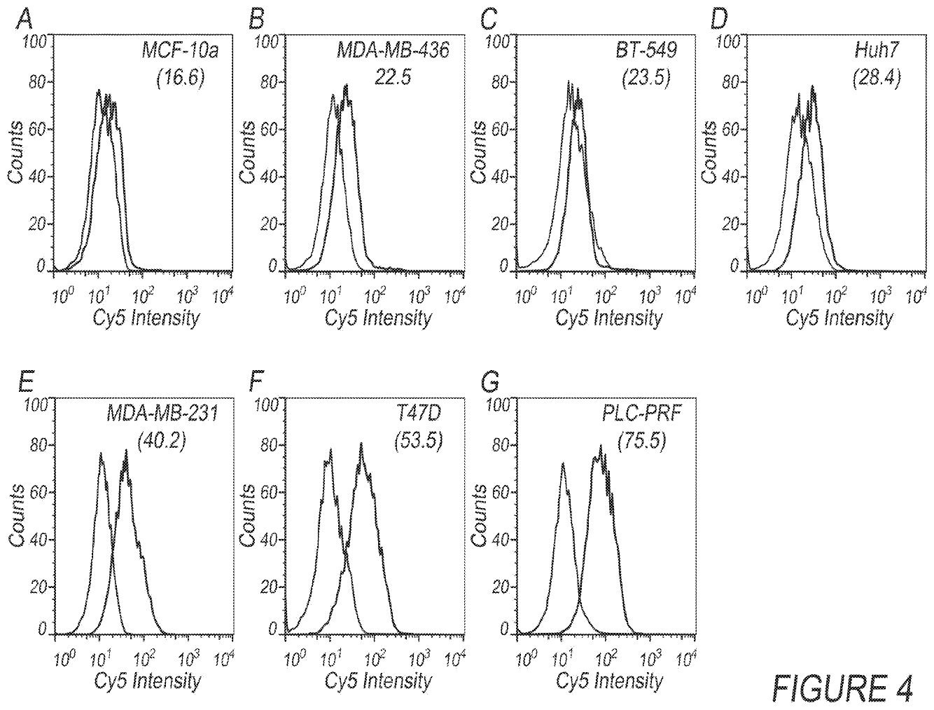

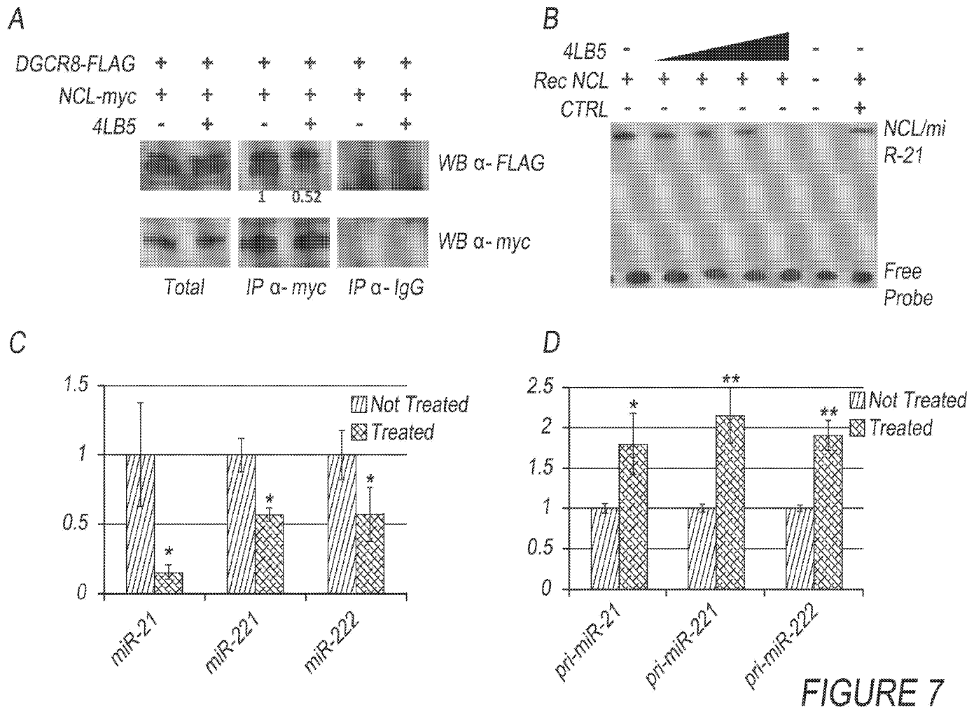

The binding of 4LB5 to surface-NCL was then assessed by ELISA using MDA-MB-231 breast cancer cells, which express high levels of surface-NCL(19). FIG. 2B shows the efficient binding of 4LB5 to the surface of these cells. To evaluate the detection limit of the ELISA performed using our scFv, the assay was performed using different amounts of MDA-MB-231 cells and different concentrations of 4LB5. As shown in FIG. 3B, at concentrations ranging from 400-600 nM 4LB5 was able to detect as low as 50 cells, compared to the negative control. However, when used at 200 nM and 50 cells were plated, 4LB5 resulted in a signal that was not significantly different from the background. To confirm that the observed binding was due to the specific interaction between 4LB5 and NCL, MDA-MB-231 cells were transfected with a control (siCTRL) or anti-NCL specific siRNAs (siNCL) and analyzed by ELISA using different concentrations of 4LB5. Abrogation of NCL expression resulted in a significant reduction of 4LB5 binding (FIG. 2C). Western blot analysis of total MDA-MB-231 cell extracts using 4LB5 as a primary antibody further confirmed that 4LB5 was able to discriminate between siCTRL- and siNCL-transfected cells like the commercial anti-NCL antibody (FIG. 3C). It was observed, by ELISA, the differential binding of 4LB5 to MDA-MB-231 cells compared to normal-like MCF-10a breast cells, expressing low levels of surface-NCL(19, 32) (FIG. 2D). Finally, the ability of 4LB5 to bind cancer cells was investigated by flow cytometry using a Cy5-labeled 4LB5 and different breast or hepatocellular carcinoma (HCC) cell lines (FIG. 4). In line with data shown in FIG. 2D, binding of Cy5-4LB5 was not detected to surface-NCL negative MCF-10a, compared to the Cy5 label alone (FIG. 7). Conversely, strong binding of 4LB5 to MDA-MB-231 and T47D (breast cancer cells) and to PLC-PRF (HCC) was detected. Reduced binding was detected on breast cancer cells BT-549 and MDA-MB-436, but also on Huh7 HCC cells (FIG. 4). These data demonstrate that 4LB5 specifically binds to various cancer cell lines but not to normal-like breast cells or following abrogation of NCL expression.

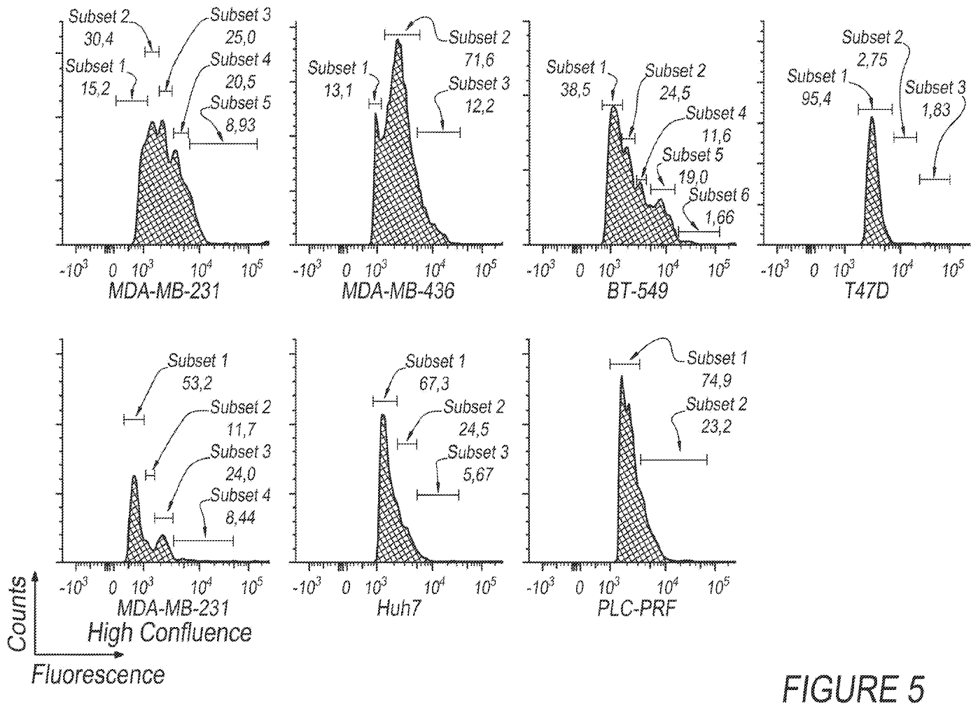

The cell lines disclosed herein were also studied to determine if they displayed heterogeneity in the levels of surface NCL. An analysis by flow cytometry using a commercially available antibody against NCL (FIG. 5) was performed. Interestingly, most of the cancer cell lines that were used display a significant heterogeneity in surface-NCL expression levels and the presence of different sub-populations in the majority of them was observed. Of note, when MDA-MB-231 cells were cultured until they reached the maximum confluence, the amount of surface-NCL-positive cells was reduced, especially those populations expressing higher levels of surface NCL. These observations are in line with previous findings indicating NCL as a useful marker of cell proliferation (Nissim A, et al. (1994) The EMBO journal 13(3):692-698).

Internalization of 4LB5 scFv

NCL is able to shuttle between the cell surface and the cytoplasm of cancer cells (Soundararajan et al. Cancer research 68(7):2358-2365; Soundararajan S, et al. (2009) Molecular pharmacology 76(5):984-9914), a property that makes NCL an attractive target for the selective delivery of anti-neoplastic drugs or pro-drugs, leaving normal cells unaffected (Koutsioumpa M & Papadimitriou E (2013)).

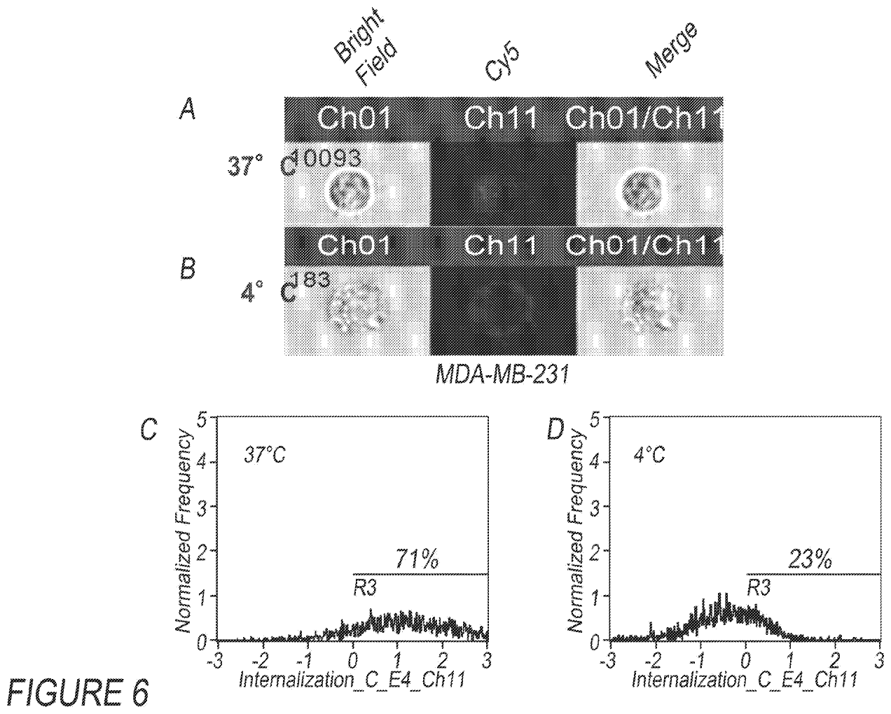

To test the ability of 4LB5 to undergo NCL-mediated endocytosis, MDA-MB-231 cells were incubated with Cy5-labeled 4LB5 (Cy5-4LB5) at 37° C. or at 4° C. for 6 h. Cells were extensively washed with PBS, harvested and analyzed by Amnis FlowSight. FIG. 6A-B shows representative bright field (left panel), Cy5 fluorescence (central panel) and merged (right panel) images of the analyzed cells. At 37° C. the majority of Cy5-4LB5 was mainly found in the cytoplasm, while at 4° C., when the active mechanisms of NCL intra-cytoplasmic re-localization are slowed, Cy5-4LB5 remained on the cell surface. Quantitative analysis (FIG. 6C-D) confirmed higher fluorescent internalization score for the cells incubated at 37° C. versus 4° C., when shuttling mechanisms are slowed.

4LB5 scFv Affects microRNA Biogenesis

Since NCL has been shown to associate with DGCR8, one of the components of the microRNA Microprocessor complex (Pichiorri F, et al. (2013) The Journal of Experimental Medicine 210(5):951-968; Shiohama et al. Experimental Cell Research 313(20):4196-4207); Pickering et al. (2011) JBC 286(51):44095-44103), the effects of 4LB5 on this interaction in HeLa cells expressing FLAG-tagged DGCR8 and myc-tagged NCL was evaluated. FIG. 7A shows that 4LB5 reduced the amount of co-immunoprecipitated NCL-myc and DGCR8-FLAG (fold-change 0.51).

NCL enhances the maturation of a subset of miRNAs (including miR-21, -221 and -222), and its inhibition by siRNAs or anti-NCL aptamers leads to down-regulation of these mature miRNAs and accumulation of their primary forms (Pichiorri F, et al. (2013) The Journal of Experimental Medicine 210(5):951-968). Therefore, the ability of NCL to bind its target miRNAs in the presence of 4LB5 by RNA-EMSA (REMSA) was assessed. As shown in FIG. 3B, 4LB5 reduced or completely abrogated the formation of the NCL/miR-21 complex, while no effect was observed when an unrelated control IgG was incubated with the complex.

Finally MDA-MB-231 breast cancer cells were treated with 4LB5 or left untreated, and RNA was extracted after 72 h. Real-Time analysis revealed that the mature forms of miR-21, miR-221, and miR-222 were significantly reduced by treatment with 4LB5 (FIG. 7C), while their primary forms (pri-miRNA) accumulated after the treatment (FIG. 7D).

Taken together, these data indicate that 4LB5 inhibits the interaction between NCL and the Microprocessor complex, impairing the maturation of NCL-associated miRNAs.

4LB5 scFv Affects Cancer Cell Viability, Proliferation, and Migration In Vitro.

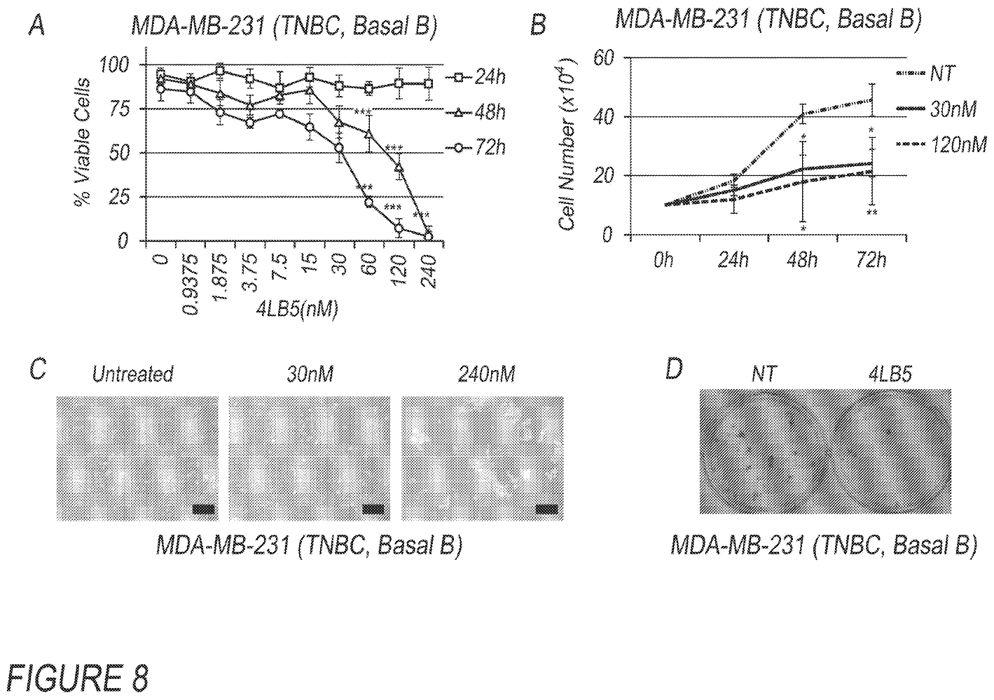

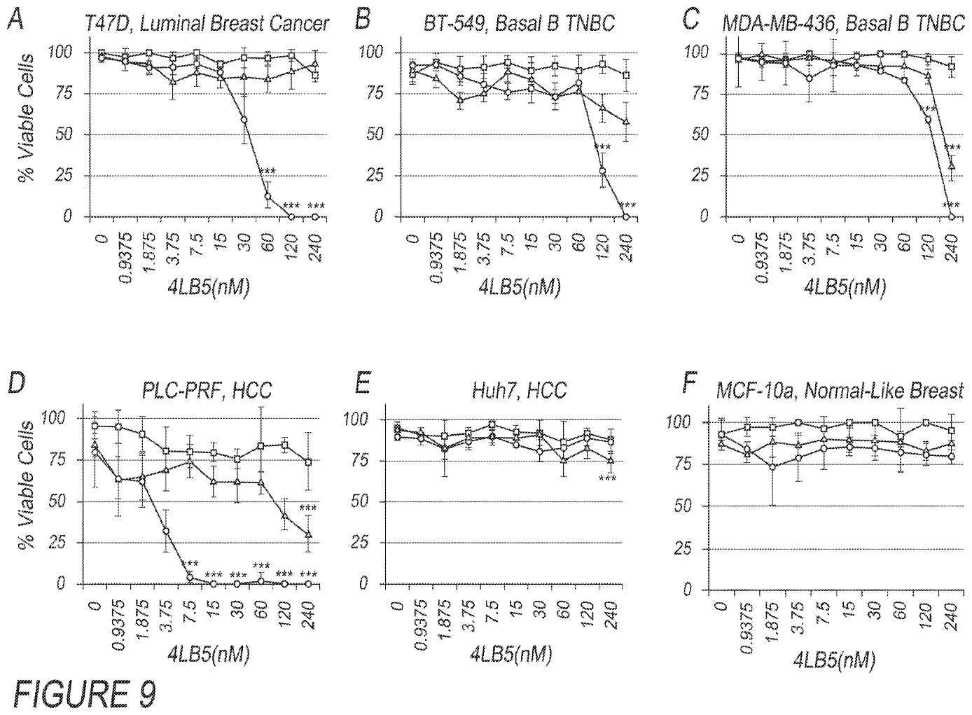

Several reports have shown that NCL inhibition by siRNAs or anti-NCL aptamers affects cell viability, proliferation, and migration (Wise J F, et al. (2013) N Blood 121(23):4729-4739, Pichiorri F, et al. (2013) The Journal of Experimental Medicine 210(5):951-968; Rosenberg J E, et al. (2013). Investigational New Drugs; Yamada et al. Leukemia Research; Schokoroy et al. PloS one 8(9):e75269; Yang X, et al. (2013) Tumour Biology; Wu J, et al. (2013) Molecular Pharmaceutics 10(10):3555-3563; Birmpas et al. Vascular Cell 4(1):21; Xu Z, et al. (2012) Journal of Neuro-Oncology 108(1):59-67). To assess the effects of 4LB5 on cell viability and proliferation, a dose-response (0.9-240 nM) experiment was performed using the scFv on MDA-MB-231 Triple Negative Breast Cancer (TNBC) cells at different time points (24, 48 and 72 h). As shown in FIG. 8A, a significant reduction of cell viability was observed after 48 and 72 h of treatment, with an IC₅₀ of ˜30 nM at 72 h. Growth curves and colony assays (FIG. 8B-D) also indicated a significant reduction in cell proliferation at 48 and 72 h of treatment. Similar results were also observed on T47D (ER+, PgR+ Luminal breast cancer) (IC₅₀˜20 nM), BT549 (Basal B TNBC) (IC₅₀˜58 nM), MDA-MB-436 (Basal B TNBC) (IC₅₀˜50 nM) and PLC-PRF (Hepatocellular Carcinoma) (IC₅₀˜3 nM) cell lines (Supplementary FIG. 9A-D), while no effect was observed on Huh7 (Hepatocellular Carcinoma) (FIG. 9E) or MCF-10a (Normal-Like Breast) cells (FIG. 9F). The different response displayed by the cancer cell lines can be dependent on several factors, including, but not limited to, the relative abundance of subpopulations expressing different levels of surface NCL (FIG. 5), the different expression levels of NCL-dependent microRNAs and the different oncogenic pathways modulated by NCL in each different cellular context.

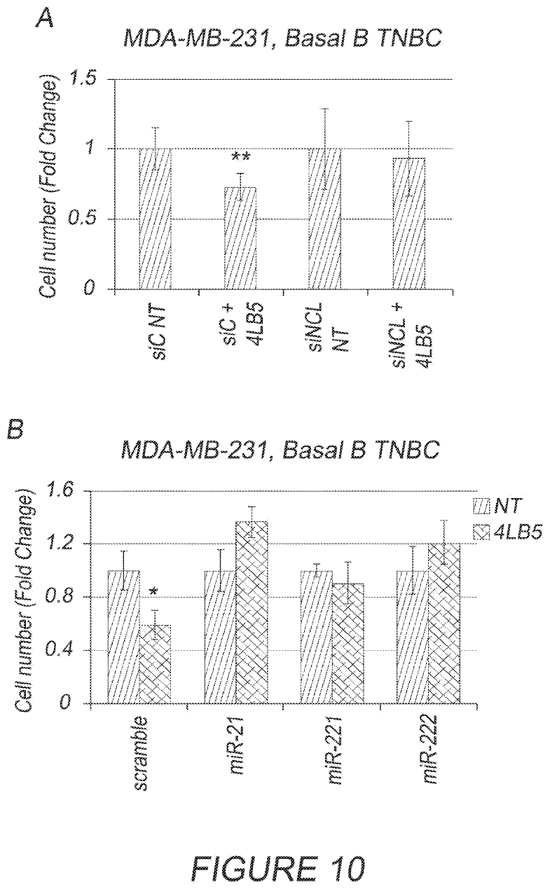

To confirm that the cytotoxic effect of 4LB5 was dependent on the specific binding of the scFv to NCL, MDA-MB-231 cells were transfected with anti-NCL siRNAs (siNCL) and treated with 4LB5. FIG. 10A shows that 4LB5 treatment failed to inhibit cell proliferation of MDA-MB-231 cells with abolished NCL expression compared to cells transfected with siNCL and not treated with the scFv. Moreover, it was also assessed whether the cytotoxic effect of NCL inhibition could be rescued by the overexpression of mature microRNAs, whose biological activity is not dependent on NCL. FIG. 10B shows that overexpression of NCL-regulated miRs, such as mature miR-21, miR-221 and miR-222, prevented 4LB5 mediated inhibition of cell proliferation.

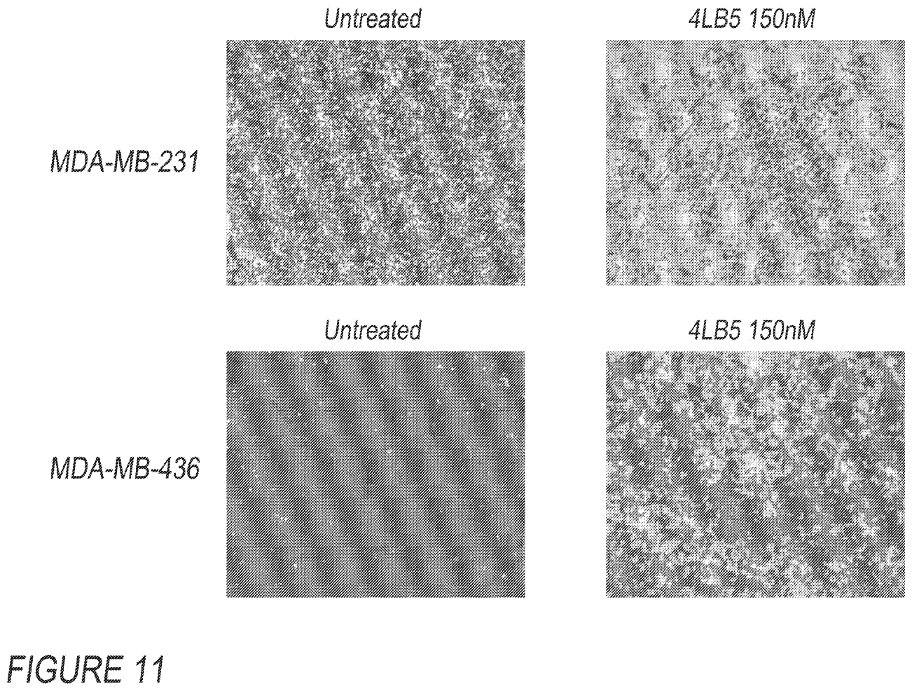

Since miR-21, -221 and -222 are extensively associated with an invasive phenotype of breast cancer (Yan L X, et al. (2011) BCR 13(1):R2; Shah M Y & Calin G A (2011) Genome Medicine 3(8):56; Di Leta G, et al. (2010) Journal of the National Cancer Institute 102(10):706-721) and NCL inhibition affects breast cancer cell migration (Pichiorri F, et al. (2013) The Journal of Experimental Medicine 210(5):951-968), it was also tested whether 4LB5 was able to inhibit this process in vitro. MDA-MB-231 and MDA-MB-436 cells were treated for 24 h with 4LB5 and then counted and re-seeded into transwell plates for additional 24 h. Compared to untreated cells, crystal violet staining revealed that 4LB5 treatment impaired cell migration in both cell lines (FIG. 11).

These observations indicate that NCL inhibition by 4LB5 significantly reduces cell viability, proliferation and migration in vitro.

4LB5 scFv Induces Apoptosis in Cancer Cells

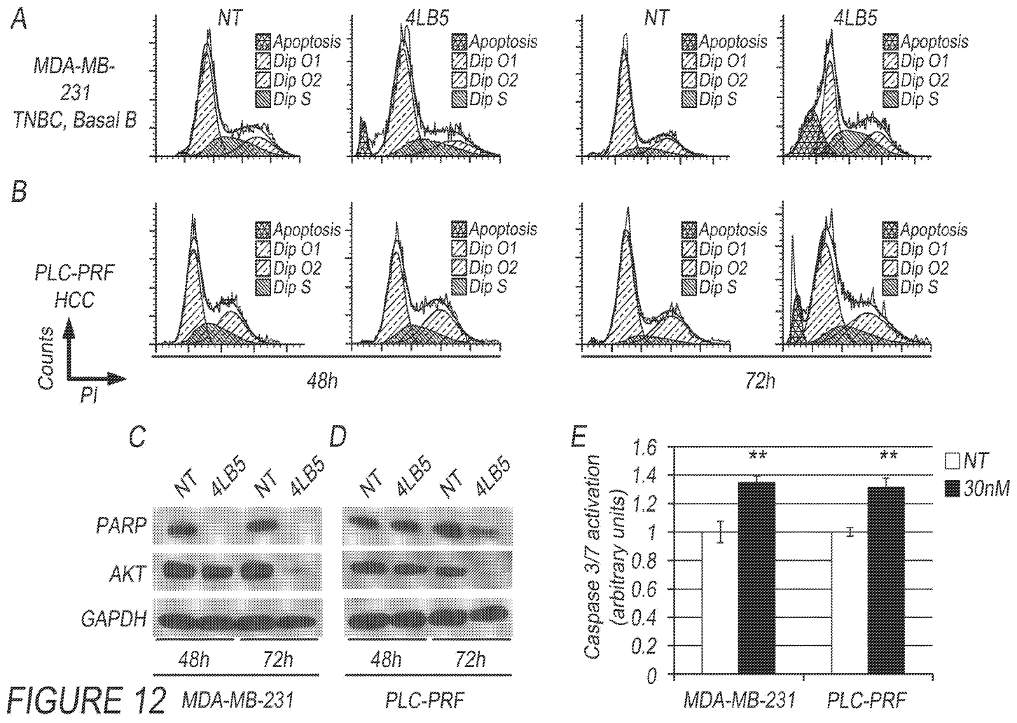

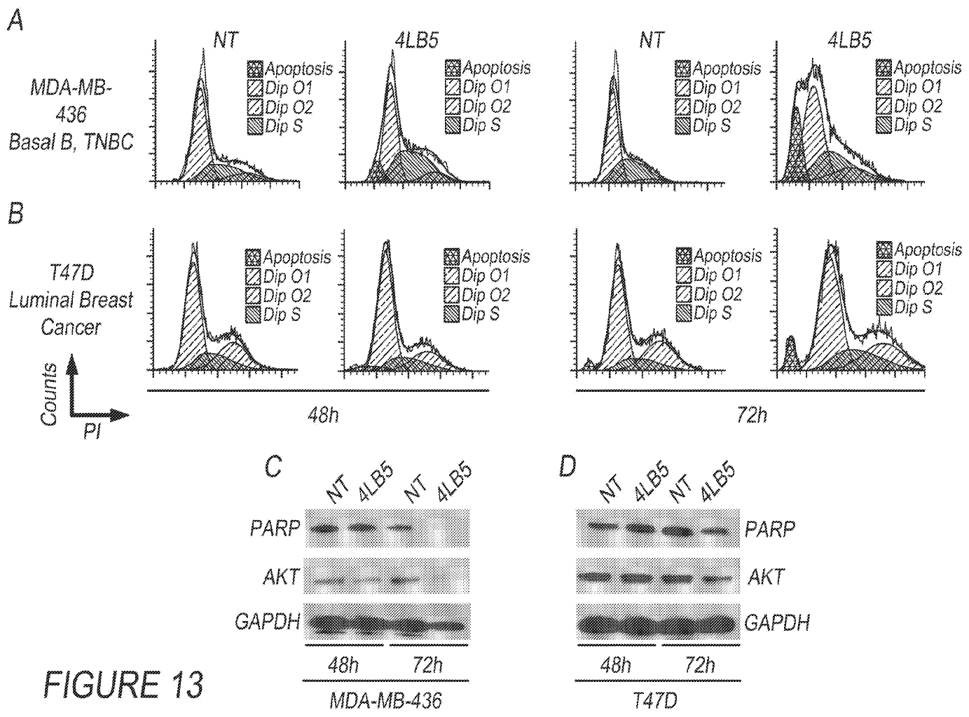

The reduced cell viability and proliferation observed following NCL inhibition by 4LB5 treatment led to experiments to determine that 4LB5 is also able to induce apoptosis. A flow-cytometric analysis of different cell lines treated with 4LB5 for 48 or 72 h was performed (FIG. 12A-B and FIG. 13A-B) and were stained with propidium iodide. In all analyzed cell lines, a sub-G1 peak, compatible with the accumulation of dead cells, was observed at 72 h of treatment with 4LB5. Western blot analysis of PARP levels confirmed the activation of apoptosis following 4LB5 treatment, resulting in inactive-PARP degradation (FIG. 12C-D and FIG. 13C-D). In the same experiment, the expression levels of AKT were also measured, a previously described anti-apoptotic factor whose expression is dependent on NCL (Abdelmohsen K. et al. (2011) Enhanced translation by Nucleolin via G-rich elements in coding and non-coding regions of target mRNAs. Nucleic acids research 39(19):8513-8530). Interestingly, NCL inhibition upon 4LB5 treatment reduced AKT levels. To further demonstrate the activation of apoptotic pathways following scFv treatment, caspase 3/7 activation in MDA-MB-231 and PLC-PRF cells treated with 4LB5 or left untreated was also measured. FIG. 12E shows a significant caspase 3/7 cleavage upon 4LB5 treatment.

Overall, these data indicate that NCL inhibition by 4LB5 treatment results in decreased cell viability and activation of programmed cell death.

4LB5 Displays Potent Anti-Tumor Activity In Vivo

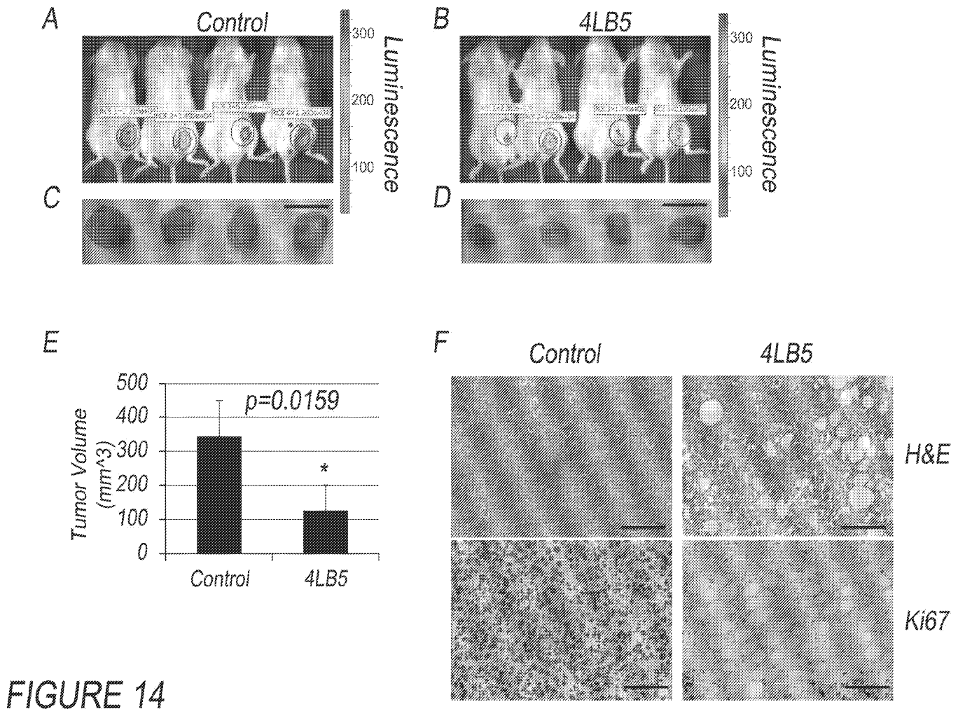

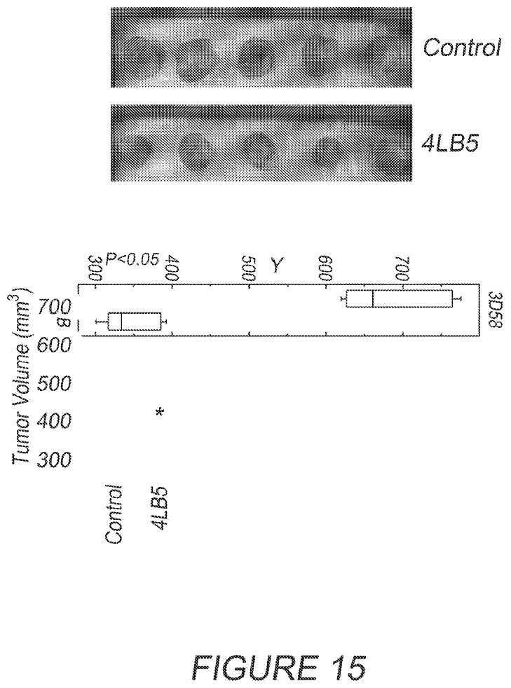

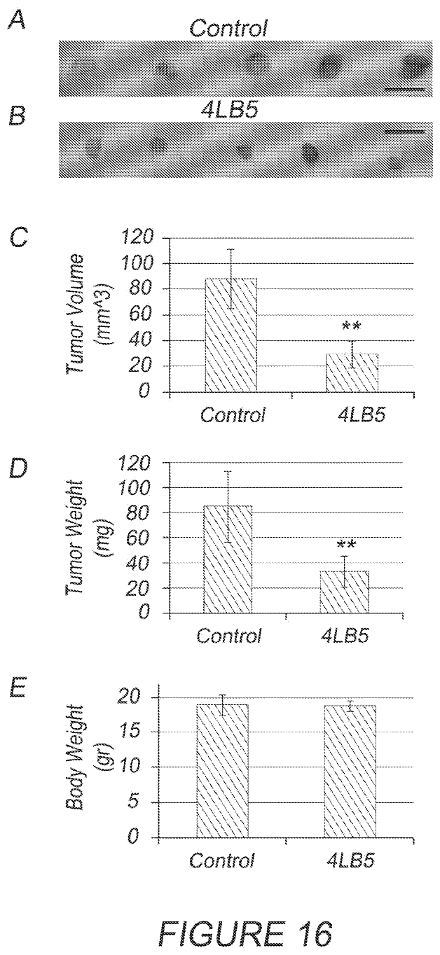

To verify the potential anti-cancer activity of anti-NCL scFv 4LB5 in vivo, an orthotopic xenograft mouse model in which Luc-MDA-MB-231 were injected into the mammary fat pad of NOD-SCID mice was employed. Two weeks after the injection, mice bearing tumors of comparable size received i.p. injections of vehicle (n=4, FIG. 14; n=5 FIG. 15) or 2 mg/kg of 4LB5 (n=4, FIG. 14; n=5 FIG. 15) twice weekly. Two weeks after the first treatment, a clear reduction of tumor size in 4LB5-treated mice was observed, in comparison with the control-treated ones, by IVIS (FIG. 14A-B). Mice were then euthanized and tumors removed for further analysis (FIG. 14C-E, FIG. 15). Compared to controls, 4LB5 treatment significantly reduced the tumor volume (p=0.0159). Interestingly, H&E staining (FIG. 14F, upper panels) showed reduced cellularity and several areas of necrosis following treatment with the scFv. In addition, Ki67 IHC staining of treated tumors indicated a reduced proliferation compared to controls (FIG. 14F, lower panels). In a different experiment, the treatment with 2 mg/kg of 4LB5 (n=5) or vehicle (n=5), twice a week for four weeks, begun at three days after the orthotopic implantation of MDA-MB-231 cells. Excised tumors (FIG. 16A-D) displayed a significant reduction in the tumor volume and weight in 4LB5-treated mice in compared to controls, while alteration of health conditions and body weight was not observed (FIG. 16E) in scFv-treated mice, showing that 4LB5 was not toxic for normal cells.

The in vivo observations clearly indicate that 4LB5 is effective in reducing the viability and proliferation of aggressive breast cancer cells in the absence of detectable side effects.

Discussion

The widely-demonstrated role of NCL in tumorigenesis suggests that inhibition of its oncogenic actions reduces tumor aggressiveness (Ishimaru D, et al. (2010) JBC 285(35):27182-27191) and several studies have proposed NCL as an ideal target for anti-neoplastic therapies in different solid and hematological malignancies (Li J, et al. (2013) Nuclear Medicine and Biology; Birmpas et al. (2012) BMC Cell Biology 13:32). Given the selective presence of NCL on cancer cells and cancer-associated endothelial cells, but not on normal cells, molecules targeting NCL might represent an effective approach for the selective delivery of drugs or toxins to tumors while minimizing side effects (Koutsioumpa et al; Wu J, et al. (2013) Molecular Pharmaceutics 10(10):3555-3563). In addition, NCL-ligands can be modified to develop novel cancer imaging and diagnostic tools (Koutsioumpa et al).

Novel anti-NCL molecules with a strong relevance in terms of efficacy and clinical pertinence for cancer therapy were identified. Taking advantage of phage-display technology, a fully human recombinant scFv, named 4LB5 was selected, which specifically binds NCL on the cell surface of cancer cells. This molecule displayed a significant ability to discriminate between cancer and normal-like breast cells.

It was demonstrated that 4LB5 treatment affects the expression of mature miR-21, -221 and -222, affecting NCL interaction with DGCR8 and halting the maturation of the primary forms of these miRNAs.

Furthermore, 4LB5 treatment of breast cancer cells, but not of normal-like breast cells, significantly reduces cell viability, proliferation and migration, and induces apoptosis, in agreement with previous in vitro studies using other anti-NCL molecules. Similar results were obtained using HCC cells expressing high levels of surface-NCL (Semenkovich et al. Biochemistry 29(41):9708-9713), showing that NCL inhibition is a valid approach for the treatment of different types of tumors.

It was also demonstrated that 4LB5 treatment reduces tumor growth in vivo in an orthotopic xenograft mouse model of breast cancer, in the absence of any evident side effect.

scFvs can be modified by one of skill in the art to obtain a compact (De Lorenzo C, et al (2004) British Journal of Cancer 91(6):1200-1204) or a full-length human immunoglobulin with the same specificity, but with a prolonged in vivo half-life and the ability to activate CDC and ADCC, combining the anti-tumoral activity of NCL inhibition with an immune response against cancer cells.

It was also shown that that 4LB5 translocates into the cytoplasm following NCL binding, suggesting its use to vehicle anti-tumoral molecules (pro-drugs, enzymes, toxins and radionuclides) directly into cancer cells, enhancing their therapeutic activity while reducing their adverse effects.

Materials and Methods

Cell Cultures and Transfections

MDA-MB-231, MDA-MB-436, BT549, T47D, Huh7 and PLC-PRF cells were cultured in RPMI with 10% FBS, L-glutamine and antibiotics. HeLa cells were cultured in DMEM with 10% FBS, L-glutamine and antibiotics (Sigma). MCF10a cells were cultured in Mammary Epithelial Cell Growth Medium (MEGMA, Lonza) supplemented with 10% FBS, bovine pituitary extract, hydrocortisone, hEGF and insulin (BulletKit, Lonza). Cell lines were purchased from the American Type Culture Collection (ATCC) and cultured in humidified atmosphere containing 5% CO₂ at 37° C. Transfection were performed by using Lipofectamine 2000 (Life Technologies) as suggested by the manufacturer.

Plasmids and siRNAs

pET15b and pET22b(+) prokaryotic expression vectors were purchased from Novagen. pHEN2 phagemid vector was described previously (Nissim A, et al. (1994) The EMBO journal 13(3):692-698).

Internalization Experiments

Subconfluent MDA-MB-231 cells were treated with 1 μg/ml of Cy5-4LB5 diluted in complete medium and cultured at 37° C. or at 4° C. for 6 h to allow the internalization of the scFv. Cells were then extensively washed with PBS, gently scraped and acquired by ImageStream (Amnis) to determine the extent of internalization. Bright field and Cy5 images were acquired and analyzed using the built-in Amnis internalization wizard.

RNA Electrophoretic Mobility Shift Assay (REMSA)

REMSA was performed using the LightShift Chemiluminescent EMSA kit (Thermo Fisher Scientific), according to the manufacturer's instructions. In brief, 1 nmol of biotinylated miR-21 were incubated with 50 ng of recombinant NCL-RBD-His6 for 30 min at room temperature. For competition experiments, recombinant proteins were pre-incubated with increasing concentrations of 4LB5 (80-650 nM) or with control IgG. Binding reactions were run on a native 7% polyacrylamide—1× TBE gel. Transfer of binding reactions to nylon membranes and detection were performed according to the manufacturer's instruction.

Quantitative Real Time PCR (qRT-PCR)

qRT-PCRs were performed using the TaqMan Fast-PCR kit (Applied Biosystems) according to the manufacturer's instructions, using the appropriate TaqMan probes for miRNA and pri-miRNA quantification, followed by detection with the 7900HT Sequence Detection System (Applied Biosystems). All reactions were performed in triplicate. Simultaneous quantification of RNU6 was used as reference for miRNA quantification. Simultaneous quantification of GAPDH mRNAs was used as reference for pri-mRNA quantification. The comparative cycle threshold (Ct) method for relative quantification of gene and miRNA expression (User Bulletin #2; Applied Biosystems) was used to determine miRNA and pri-miRNA, expression levels.

Cell Viability and Growth Assays

For viability assays, 1×10⁵ cells were plated in 12-well plates and treated with the indicated amounts (1-240 nM) of 4LB5. At the indicated time points, cells were harvested, mixed 1:1 with Trypan blue and counted using a hemacytometer. The percentage of viable cells is reported. Inhibitor concentration 50 (IC50) was evaluated using the Prism 6.0 software (Graphpad software).

For cell growth curves, 1×10⁵ cells were plated in 12-well plates and treated with the indicated amounts (30-120 nM) of 4LB5. Cells were harvested every 24 h for 3 days and counted as described above. Total cell numbers were reported.

Colony Assay

For colony assay experiments, 200 MDA-MB-231 cells were plated in 12-well plates and treated with 30 nM 4LB5 in complete medium for 72 h. Then, cells were replenished with complete medium without 4LB5 and allowed to grow for 7 additional days, to allow the formation of the colonies. Cells were then fixed with 1% glutaraldehyde in PBS and stained with crystal violet.

Migration Assay

Transwell insert chambers with 8-μm porous membrane (Greiner-Bio-One) were used for migration assay. MDA-MB-231 and MDA-MB-436 cells were treated with 150 nM 4LB5 for 24 h, harvested and 5×10⁴ viable cells were added to the top chamber in serum-free media plus 150 nM 4LB5. The lower chamber was filled with complete media. Chambers were incubated for 24 h at 37° C. in humidified atmosphere. Cells on the top of the chamber were then removed using a cotton swab, while migrated cells were fixed in 1% glutaraldehyde-PBS, stained with crystal violet and visualized under a phase-contrast microscope (E200, Nikon).

In Vivo Experiments

For the establishment of xenograft models, 2×10⁶ viable Luc+ MDA-MB-231 cells were injected into the fourth left-side mammary fat pad of female NOD-SCID mice (NOD/ShiLtSz; Charles River). Three days or two weeks after tumor cell inoculation, mice were treated twice a week with i.p. injections of 4LB5 (2 mg/kg) or control buffer (25 mM imidazole in PBS) diluted in 100 μl PBS. Tumor size was assessed every 7 d by bioluminescence imaging, as described below. After 4 weeks of treatment, mice were analyzed by bioluminescence images and then euthanized. For in vivo bioluminescence analysis, mice were injected with 75 mg/kg Luciferin (Xenogen), and tumor growth was detected by bioluminescence at 20 min after the injection. The home-built bioluminescence system used an electron multiplying charge-coupled device (IVIS-200, Perkin-Elmer) with an exposure time of 30 s and an electron multiplication gain of 500 voltage gain×200, 5-by-5 binning, and with background subtraction. The tumor size was measured using a caliper, and the volume was calculated in cubed millimeters using the formula L×W×H.

Selection of scFv Phage Clones

Phagemid particles were rescued with M13-K07 (Life Technologies) from the Griffin.1 library, as previously described (De Lorenzo C, et al. (2004) A human, compact, fully functional anti-ErbB2 antibody as a novel antitumour agent. British journal of cancer 91(6):1200-1204). For each round of selection, phages (10¹³ cfu) were blocked with 5% Non-Fat Dry Milk (Biorad) in PBS for 15 min. Polypropylene tubes were coated with recombinant NCL-RBD in PBS at a concentration of 20 μg/ml in the first four round of selection and at a concentration of 10 μg/ml in the fifth round. Blocked phages were incubated for 16 h at 4° C. in rotation in the coated tubes, and then elutes with 50 mM citric acid (pH 2.5) in PBS for 5 min, and then neutralized with 1M Tris-HCl (pH 7.4). Recovered phages were amplified by infecting E. coli TG1 bacterial strain to prepare phages for the next round of selection. Phage screening was carried out by ELISA as described below. Cultures of E. coli SF110 bacterial strain, previously infected with selected phage clones, were grown at 37° C. in 2×TY medium containing 100 μg/ml ampicillin and 1% glucose, until 0.D.=600 nm was reached. Cells were centrifuged at 6,000 rpm for 15 min and resuspended in glucose-free medium. The expression of soluble scFv was induced by the addition of isopropyl-1-thio-β-D-galactopyranoside (Calbiochem) to a final concentration of 1 mM in the cell culture, which was then grown at room temperature overnight. Cells were harvested by centrifugation at 6,000 rpm for 15 min, and a periplasmic extract was obtained by resuspending cells in B-PER buffer (Thermo Pierce), according to the manufacturer's recommendations.

Purification of Recombinant Proteins

pET15b-NCL-RBD-His6 was expressed in E. coli BL21(DE3) (Agilent Technologies) bacterial cells as soluble protein following IPTG induction and purified using nickel affinity chromatography (Qiagen) according to the manufacturer's instructions.

Recombinant 4LB5 scFv showed reduced solubility and for this reason was extracted from the insoluble fraction of pET22b(+)-4LB5-transformed BL21(DE3) bacterial cells. The fraction was solubilized with 7M urea, 2M thiourea, 20 mM Tris, pH 8.0 and 50 mM NaCl and mixed overnight at room temperature. The solution was centrifuged at 12,000 rpm for 1 h to pellet. Supernatant was removed and the resulting pellet was re-solubilized with the same buffer with the addition of 0.5 mM Aminosulfobetaine-14 and 0.1% IQEPAL for 48 h and centrifuged at 12,000 rpm for 1 hour. The supernatant was diluted to one-half concentration and applied to a pretreated tandem Q-HiTrap/S-HiTrap at 0.5 ml/min. The flowthrough was collected and the column washed with 10 column volumes of 3.5M urea, 2M thiourea, 10 mM Tris, pH 8.0, and 25 mM NaCl. The columns were eluted with wash buffer with the addition of 1M NaCl. Flow through was applied to a 5 mL Hitrap column charged with nickel sulfate solution and prepared with 3.5M urea, 2M thiourea, 10 mM Tris, pH 8.0, 100 mM NaCl and 20 mM of imidazole. The protein solution, diluted with 3.5 mM urea buffer, was applied to the column and washed with 10 column volumes of buffer. The column was then washed slowly with decreasing urea concentrations to promote folding in 10 mM Tris pH 8.0, 100 mM NaCl and 10% glycerol to support the solubility. The column was then washed in PBS and eluted with PBS with 250 mM imidazole. Protein purity and quantification were assessed by SDS-PAGE and Coomassie blue staining.

ELISA

Phages and soluble scFvs, prepared as described above, were evaluated for their affinity to bind NCL-RBD by ELISA. Flat-bottom 96-well plates were coated with 20 μg/ml of recombinant NCL-RBD in 2% Non Fat dry milk (NFDM) in PBS. Phages or soluble scFvs were added to the plates in 2% NFDM and incubated for 2 h at room temperature. Plates were washed with PBS and incubated with HRP-conjugated anti-M13 antibody (Amersham) for 1 h, washed again and incubated with TMB reagent (Sigma) for 10 min before quenching with an equal volume of 1M HCl.

For cell ELISA, 1×10⁴ MDA-MB-231 or MCF10a cells were incubated in round-bottom 96-well plates with different concentrations (0-600 nM) of 4LB5 in 2% NFDM for 2 h at room temperature with gentle agitation. Plates were then centrifuged and cell pellets were washed with PBS and incubated with HRP-conjugated anti-penta-His antibody (Qiagen) for 1 h at room temperature. Following additional washes, TMB reagent (Sigma) was added for 10 min before quenching with an equal volume of 1M HCl. ELISA plates were read (A₄₅₀) using a Spectramax 340 microtiter plate reader (Molecular Devices).

Surface Plasmon Resonance (SPR)

The SPR analyses were performed at 25° C. on a BIAcore 3000 instrument (Biacore AB) equipped with research-grade CM5 sensor chips (Biacore AB). The running buffer was HBS-EP (10 mm Hepes, 0.15 m NaCl, 3.4 mm EDTA and 0.005% surfactant P20 at pH 7.4). Coupling reagents, N-hydroxysuccinimide, 1-ethyl-3-(3-dimethylaminopropyl)-carbodiimide hydrochloride, ethanolamine hydrochloride and HBS-EP running buffer were purchased from Biacore AB. To measure the binding properties of 4LB5 to the NCL-RBD, recombinant NCL-RBD was immobilized onto the surface of sensor chip CM5 using the standard amine coupling chemistry. Typically, 350 and 700 RU of NCL-RBD were immobilized onto the sensor surface. Binding curves were recorded by injecting 4LB5 (5-500 nM) over the immobilized NCL-RBD at a constant flow rate of 50 μL·min⁻¹. Association and dissociation phases were recorded for 300 and 1200 s, respectively. The rate constants of the interactions described above were calculated by non-linear analysis of the association and dissociation curves using SPR kinetic evaluation software BIAevaluation (Biacore AB), fitting data to the 1:1 Langmuir binding model. The equilibrium dissociation constants (K_{D}) were calculated from the values of the association rate constant kₐ and dissociation rate constant k_{d}, according to the thermodynamic relationship K_{D}×k_{d}/kₐ.

Western Blot, Co-Immunoprecipitation Experiments and Antibodies

Periplasmic extracts were obtained as previously described (De Lorenzo C, et al. (2004) A human, compact, fully functional anti-ErbB2 antibody as a novel antitumour agent. British journal of cancer 91(6):1200-1204) using the B-PER extraction buffer (Thermo Scientific) supplemented with protease inhibitors (Calbiochem). Insoluble fractions were resuspended in 8M urea before SDS-PAGE and Coomassie blue staining or western blot using standard procedure. Eukaryotic protein extracts were obtained using 1% NP-40, 1 mm EDTA, 50 mm TrisHCl, pH 7.5, and 150 mm NaCl, supplemented with complete protease and phosphatase inhibitors (Calbiochem). Protein extracts were subjected to SDS-PAGE, electroblotted onto Nitrocellulose membranes, and probed with antibodies as indicated, according to standard procedures. For immunoprecipitation experiments, subconfluent HeLa cells were transfected with the FLAG-DGCR8 and NCL-Myc expression vectors. 24 h after transfection, cells were treated with 120 nM 4LB5 or control buffer (12.5 mM imidazole in PBS) for an additional 24 h. Proteins were extracted as described above, and 2 mg of total protein extracts were immunoprecipitated with anti-FLAG-M2 resin (Sigma) resin overnight at 4° C. with rotation. Immunoprecipitates were washed as suggested by the manufacturer, subjected to SDS-PAGE, electroblotted onto Nitrocellulose membranes, and probed with antibodies as indicated. Used antibodies were anti-penta-His antibody (Qiagen), anti-PARP, anti-AKT, anti-GAPDH (Cell Signaling), anti-NCL, anti-Myc-tag and anti-Tubulin (Santa Cruz Biotechnology).

Cy5 Labeling and Flow-Cytometry Analysis

4LB5 was Cy5 labeled using the LYNX Rapid Cy5 Antibody conjugation kit (AbD Serotec) according to manufacturer's instructions. Briefly, 1 mg of 4LB5 was incubated with modifier reagent and the LYNX lyophilized mix overnight at room temperature. The reaction was then stopped using the quencher reagent.

For flow-cytometry analysis, cells were harvested and incubated with 1 μg/ml of Cy5-4LB5 diluted in PBS containing 2% FBS on ice. Cells were then washed and analyzed with a FACS-Calibur flow cytometer (Becton Dickinson).

Cell Cycle Analysis

For cell cycle analysis, 24 h following the seeding, cells were treated with 240 nM 4LB5. Cells were harvested at different time-points, fixed and permeabilized with ice-cold 70% ethanol, treated with RNase-I (invitrogen) and stained with 10 mg/ml of propidium iodide. Cells were sorted on a FACS-Calibur flow cytometer (Becton Dickinson), and the results were analyzed with ModFit software, 3.2 version (Verity Software House).

Caspase Activation Assay

Apoptosis activation was quantified by measuring caspase 3 and 7 activation 48 h following 4LB5 treatment, using Caspase-Glo 3/7 assay (Promega) according to the manufacturer's instructions on a Bio-Tek Synergy HT multi-detection microplate reader.

Paraffin-Embedded Tissue Staining

Xenograft tumor samples were fixed in 10% neutral-buffered formalin, processed, embedded in paraffin, and sectioned at 4 μm. Hematoxylin and eosin staining was performed according to standard procedures. For immunohistochemical staining, slides with specimens were placed in a 60° C. oven for 1 h, cooled, deparaffinized, and rehydrated through xylene and graded ethanol solutions to water. All slides were placed for 5 min in a 3% hydrogen peroxide solution in water to block the endogenous peroxidase. Antigen retrieval was performed by HIER, in which the slides were placed in a citric acid solution, pH 6.1, for 25 min at 96° C. and cooled for 15 min in solution. Sections were then treated with primary antibodies for Ki-67, followed by biotinylated secondary antibodies and the DAB chromogen.

Statistical Analysis

Student's t test was used to determine the statistical significance (indicated as p-value) for each experiment. All error bars represent the SD of the mean. Data were considered statistically significant for p<0.05, at least.

### REFERENCES

1. Bugler B, Caizergues-Ferrer M, Bouche G, Bourbon H, & Amalric F (1982) Detection and localization of a class of proteins immunologically related to a 100-kDa nucleolar protein. European journal of biochemistry/FEBS 128(2-3):475-480.
2. Warner J R (1990) The nucleolus and ribosome formation. Current opinion in cell biology 2(3):521-527.
3. Borer R A, Lehner C F, Eppenberger H M, & Nigg E A (1989) Major nucleolar proteins shuttle between nucleus and cytoplasm. Cell 56(3):379-390.
4. Mongelard F & Bouvet P (2007) Nucleolin: a multiFACeTed protein. Trends in cell biology 17(2):80-86.
5. Srivastava M & Pollard H B (1999) Molecular dissection of nucleolin's role in growth and cell proliferation: new insights. FASEB journal: official publication of the Federation of American Societies for Experimental Biology 13(14):1911-1922.
6. Ridley L, et al. (2008) Multifactorial analysis of predictors of outcome in pediatric intracranial ependymoma. Neuro-oncology 10(5):675-689.
7. Hovanessian A G, et al. (2000) The cell-surface-expressed nucleolin is associated with the actin cytoskeleton. Experimental cell research 261(2):312-328.
8. Christian S, et al. (2003) Nucleolin expressed at the cell surface is a marker of endothelial cells in angiogenic blood vessels. The Journal of cell biology 163(4):871-878.
9. Otake Y, et al. (2007) Overexpression of nucleolin in chronic lymphocytic leukemia cells induces stabilization of bcl2 mRNA. Blood 109(7):3069-3075.
10. Chen C Y, et al. (2000) Nucleolin and YB-1 are required for JNK-mediated interleukin-2 mRNA stabilization during T-cell activation. Genes & development 14(10):1236-1248.
11. Abdelmohsen K, et al. (2011) Enhanced translation by Nucleolin via G-rich elements in coding and non-coding regions of target mRNAs. Nucleic acids research 39(19):8513-8530.
12. Reyes-Reyes E M & Akiyama S K (2008) Cell-surface nucleolin is a signal transducing P-selectin binding protein for human colon carcinoma cells. Experimental cell research 314(11-12):2212-2223.
13. Tate A, et al. (2006) Met-Independent Hepatocyte Growth Factor-mediated regulation of cell adhesion in human prostate cancer cells. BMC cancer 6:197.
14. Wise J F, et al. (2013) Nucleolin inhibits Fas ligand binding and suppresses Fas-mediated apoptosis in vivo via a surface nucleolin-Fas complex. Blood 121(23):4729-4739.
15. Abdelmohsen K & Gorospe M (2012) RNA-binding protein nucleolin in disease. RNA biology 9(6):799-808.
16. Tayyari F, et al. (2011) Identification of nucleolin as a cellular receptor for human respiratory syncytial virus. Nature medicine 17(9):1132-1135.
17. Bartel D P (2004) MicroRNAs: genomics, biogenesis, mechanism, and function. Cell 116(2):281-297.
18. Pillai R S, Bhattacharyya S N, Filipowicz W (2007) Repression of protein synthesis by miRNAs: how many mechanisms? Trends in cell biology 17(3):118-126.
19. Pichiorri F, et al. (2013) In vivo NCL targeting affects breast cancer aggressiveness through miRNA regulation. The Journal of experimental medicine 210(5):951-968.
20. Rao X, et al. (2011) MicroRNA-221/222 confers breast cancer fulvestrant resistance by regulating multiple signaling pathways. Oncogene 30(9):1082-1097.
21. Pogribny I P, et al. (2010) Alterations of microRNAs and their targets are associated with acquired resistance of MCF-7 breast cancer cells to cisplatin. International journal of cancer. Journal international du cancer 127(8):1785-1794.
22. Anastasov N, et al. (2012) Radiation resistance due to high expression of miR-21 and G2/M checkpoint arrest in breast cancer cells. Radiation oncology 7:206.
23. Mei M, et al. (2010) Downregulation of miR-21 enhances chemotherapeutic effect of taxol in breast carcinoma cells. Technology in cancer research & treatment 9(1):77-86.
24. Bates P J, Laber D A, Miller D M, Thomas S D, & Trent J O (2009) Discovery and development of the G-rich oligonucleotide AS1411 as a novel treatment for cancer. Experimental and molecular pathology 86(3):151-164.
25. Koutsioumpa M & Papadimitriou E (2013) Cell Surface Nucleolin as A Target for Anti-Cancer Therapies, Recent patents on anti-cancer drug discovery.
26. Destouches D, et al. (2008) Suppression of tumor growth and angiogenesis by a specific antagonist of the cell-surface expressed nucleolin. PloS one 3(6):e2518.
27. El Khoury D, et al. (2010) Targeting surface nucleolin with a multivalent pseudopeptide delays development of spontaneous melanoma in RET transgenic mice. BMC cancer 10:325.
28. Krust B, El Khoury D, Nondier I, Soundaramourty C, & Hovanessian A G (2011) Targeting surface nucleolin with multivalent HB-19 and related Nucant pseudopeptides results in distinct inhibitory mechanisms depending on the malignant tumor cell type. BMC cancer 11:333.
29. De Lorenzo C & D'Alessio G (2008) From immunotoxins to immunoRNases. Current pharmaceutical biotechnology 9(3):210-214.
30. Marks J D, et al. (1991) By-passing immunization. Human antibodies from V-gene libraries displayed on phage. Journal of molecular biology 222(3):581-597.
31. Nissim A, et al. (1994) Antibody fragments from a ‘single pot’ phage display library as immunochemical reagents. The EMBO journal 13(3):692-698.
32. Soundararajan S, Chen W, Spicer E K, Courtenay-Luck N, & Fernandes D J (2008) The nucleolin targeting aptamer AS1411 destabilizes Bcl-2 messenger RNA in human breast cancer cells. Cancer research 68(7):2358-2365.
33. Derenzini M, Sirri V, Trere D, & Ochs R L (1995) The quantity of nucleolar proteins nucleolin and protein B23 is related to cell doubling time in human cancer cells. Laboratory investigation; a journal of technical methods and pathology 73(4):497-502.
34. Soundararajan S, et al. (2009) Plasma membrane nucleolin is a receptor for the anticancer aptamer AS1411 in MV4-11 leukemia cells. Molecular pharmacology 76(5):984-991.
35. Shiohama A, Sasaki T, Noda S, Minoshima S, & Shimizu N (2007) Nucleolar localization of DGCR8 and identification of eleven DGCR8-associated proteins. Experimental cell research 313(20):4196-4207.
36. Pickering B F, Yu D, & Van Dyke M W (2011) Nucleolin protein interacts with microprocessor complex to affect biogenesis of microRNAs 15a and 16. The Journal of biological chemistry 286(51):44095-44103.
37. Rosenberg J E, et al. (2013) A phase II trial of AS1411 (a novel nucleolin-targeted DNA aptamer) in metastatic renal cell carcinoma. Investigational new drugs.
38. Yamada T, Park C S, Shen Y, Rabin K R, & Lacorazza H D (2013) G0S2 inhibits the proliferation of K562 cells by interacting with nucleolin in the cytosol. Leukemia research.
39. Schokoroy S, Juster D, Kloog Y, & Pinkas-Kramarski R (2013) Disrupting the oncogenic synergism between nucleolin and Ras results in cell growth inhibition and cell death. PloS one 8(9):e75269.
40. Yang X, et al. (2013) Cell surface nucleolin is crucial in the activation of the CXCL12/CXCR4 signaling pathway. Tumour biology: the journal of the International Society for Oncodevelopmental Biology and Medicine.
41. Wu J, et al. (2013) Nucleolin targeting AS1411 modified protein nanoparticle for antitumor drugs delivery. Molecular pharmaceutics 10(10):3555-3563.
42. Birmpas C, Briand J P, Courty J, & Katsoris P (2012) The pseudopeptide HB-19 binds to cell surface nucleolin and inhibits angiogenesis. Vascular cell 4(1):21.
43. Xu Z, et al. (2012) Knocking down nucleolin expression in gliomas inhibits tumor growth and induces cell cycle arrest. Journal of neuro-oncology 108(1):59-67.
44. Yan L X, et al. (2011) Knockdown of miR-21 in human breast cancer cell lines inhibits proliferation, in vitro migration and in vivo tumor growth. Breast cancer research: BCR 13(1):R2.
45. Shah M Y & Calm G A (2011) MicroRNAs miR-221 and miR-222: a new level of regulation in aggressive breast cancer. Genome medicine 3(8):56.
46. Di Leda G, et al. (2010) MicroRNA cluster 221-222 and estrogen receptor alpha interactions in breast cancer. Journal of the National Cancer Institute 102(10):706-721
47. Ishimaru D, et al. (2010) Mechanism of regulation of bcl-2 mRNA by nucleolin and A+U-rich element-binding factor 1 (AUF1). The Journal of biological chemistry 285(35):27182-27191.
48. Li J, et al. (2013) Aptamer imaging with Cu-64 labeled AS1411: Preliminary assessment in lung cancer. Nuclear medicine and biology.
49. Birmpas C, Briand J P, Courty J, & Katsoris P (2012) Nucleolin mediates the antiangiogenesis effect of the pseudopeptide N6L. BMC cell biology 13:32.
50. Semenkovich C F, Ostlund R E, Jr., Olson M O, & Yang J W (1990) A protein partially expressed on the surface of HepG2 cells that binds lipoproteins specifically is nucleolin. Biochemistry 29(41):9708-9713.
51. De Lorenzo C, et al. (2004) A human, compact, fully functional anti-ErbB2 antibody as a novel antitumour agent. British journal of cancer 91(6):1200-1204.

1. An antibody fragment which specifically binds nucleolin (NCL).

2. The antibody fragment of claim 1, wherein the fragment is a single chain variable fragment (scFv).

3. The antibody fragment of claim 1, wherein the antibody fragment specifically binds to RNA binding domain (RBD) of nucleolin.

4. The antibody fragment of claim 3, wherein the fragment binds only to RBD of nucleolin.

5. A nucleic acid sequence from which may be expressed the antibody fragment of claim 1.

6. A vector comprising a nucleic acid sequence according to claim 5.

7. An isolated cell that produces the antibody fragment of claim 1.

8. A composition comprising the antibody fragment of claim 1 and a pharmaceutically acceptable carrier.

9. A composition suitable for treatment of cancer comprising a therapeutically effective amount of an antibody fragment according to claim 1.

10. A composition suitable for treatment of infection or other non-malignant diseases, comprising a therapeutically effective amount of an antibody fragment according to claim 1.

11. The composition of claim 9, wherein said antibody fragment is, directly or indirectly, associated with or linked to an effector moiety having therapeutic activity, and the composition is suitable for the treatment of cancer or infectious disease.

12. The composition of claim 11, wherein said effector moiety is a radionuclide, therapeutic enzyme, anti-cancer drug, cytokine, cytotoxin, antibiotic, or anti-proliferative agent.

13. The composition of claim 11, wherein the effector moiety is a nucleic acid.

14. The composition of claim 13, wherein the nucleic acid is microRNA.

15. A composition suitable for the in vivo or in vitro detection of cancer comprising a diagnostically effective amount of an antibody fragment according to claim 1.

16. The composition of claim 15, wherein said antibody fragment is, directly or indirectly, associated with or linked to a detectable label, and the composition is suitable for detection of cancer.

17. The composition of claim 16, wherein the detectable label is a radionuclide or an enzyme.

18. A method of in vivo immunodetection of NCL-expressing cancer cells in a mammal comprising a step of administering to the mammal a diagnostically effective amount of a composition according to claim 13.

19. The method of claim 18, wherein said immunodetection is in vivo tumor imaging.

20. A kit comprising the antibody fragment of claim 1 and instructions for its use.

21-37. (canceled)
